(19) United States
(12) Patent Application Publication
(10) Pub. No.: US 2002/0107266 A1
(43) Pub. Date: Aug. 8, 2002

(54) Compounds, compositions and methods for treatment of parasitic infections

(76) Inventors: Marguerita Lim-Wilby, La Jolla, CA (US); Joseph Edward Semple, San Diego, CA (US); Michael I. Weinhouse, Escondido, CA (US); Erick A. Goldman, Berkeley, CA (US); Gian L. Araldi, Quincy, MA (US)

Correspondence Address: HELLER EHRMAN WHITE & MCAULIFFE LLP, 4250 EXECUTIVE SQ, 7TH FLOOR, LA JOLLA, CA, 92037, US

(21) Appl. No.: 10/014,171

(22) Filed: Dec. 11, 2001

Related U.S. Application Data
(60) Provisional application No. 60/255,221, filed on Dec. 12, 2000.

(51) Int. Cl. A61K 31/4439; A61K 31/4172; A61K 31/325; A61K 31/18

(52) U.S. Cl. 514/339; 514/341; 514/400; 514/397; 514/602; 514/480; 514/489; 548/314.1; 548/339.1; 560/33; 564/91

(57) ABSTRACT

Compounds and pharmaceutical compositions useful as anti-parasitic agents agents, particularly in the treatment, prevention or amelioration of one or more symptoms of malaria or Chagas' disease, are provided. In particular, methods of modulating the activity of falcipain or cruzain, preferably inhibiting falcipain or cruzain, with the compounds and compositions are provided.

### RELATED APPLICATIONS

Benefit of priority is claimed under 35 U.S.C. §119(e) to U.S. application Ser. No. 60/255,221, filed Dec. 12, 2000, to Lim-Wilby et al., entitled “COMPOUNDS, COMPOSITIONS AND METHODS FOR TREATMENT OF PARASITIC INFECTIONS.” The disclosure of the above-referenced application is incorporated by reference herein in its entirety.

### FIELD OF THE INVENTION

Provided herein are compounds and pharmaceutical compositions useful as anti-parasitic agents. In particular, the compounds and pharmaceutical compositions are active in assays that measure inhibition of parasitic proteases, including falcipain and cruzain. Methods of treatment, prevention, or amelioration of one or more symptoms of parasitic infections, particularly malaria and Chagas' disease, are also provided.

### BACKGROUND OF THE INVENTION

### Malaria

Malaria infects hundreds of millions of people annually, and infections with Plasmodium falciparum, the most virulent human malaria parasite, cause more than one million deaths per year. The incidence of malaria infection is not decreasing in most malaria-endemic areas of the world, despite extensive control efforts. In some areas, the incidence of malaria infection is increasing. Malaria parasites are becoming increasingly resistant to known therapies, posing greater risk of disease and death.

It is well known that degradation of hemoglobin is essential for growth and development of erythrocytic malaria parasites. It is believed that the degraded hemoglobin provides free amino acids for parasite protein synthesis. Several proteases that contribute to hemoglobin degradation in the parasitic food vacuole, including falcipain and plasmepsins I and II, have been identified and characterized.

Falcipain is a trophozoite cysteine protease isolated from P. falciparum that has been shown to degrade denatured and native hemoglobin in vitro (see, e.g., Rosenthal (1998) Emerging Infectious Diseases 4(1):49-57). The acid pH optimum, substrate specificity, and inhibitor sensitivity of falcipain indicate that it is a member of the papain family of cysteine proteases. Specific inhibitors of falcipain have been shown to block hemoglobin degradation and to prevent parasite development.

To date, relatively few inhibitors of falcipain are known (see, e.g., Rosenthal et al. (1996) Antimicrob. Agents Chemother. 40(7):1600-1603; Dominguez et al. (1 997) J. Med. Chem. 40:2726-2732; Li et al. (1996) Bioorg. Med. Chem. 4(9):1421-1427; Ring et al. (1993) Proc. Natl. Acad. Sci. USA 90:3583-3587). Those that are known suffer from in vivo toxicity, limiting their utility as therapeutic agents. Thus, there is a need for inhibitors of falcipain for use as therapeutic agents in treatment of malaria, particularly for treatment of P. falciparum induced malaria.

Therefore, it is an object herein to provide compounds, compositions and methods for modulating the activity of falcipain, particularly of inhibiting falcipain. It is a further object herein to provide compounds and compositions for treatment, prevention, or amelioration of one or more symptoms of malaria, particularly for treatment, prevention, or amelioration of one or more symptoms of P. falciparum induced malaria.

### Chagas' Disease

Chagas' disease results from infection with the protozoan parasite Trypanosoma cruzi and is the leading cause of heart disease in Latin America. Over sixteen million people are infected and over nine million are at risk. Acute Chagas' disease results in myocarditis in approximately 60% of patients with an estimated 9% mortality rate in endemic areas. Most chagasic patients die from heart failure associated with cardiomyopathy during the chronic phase of the disease.

Current therapies for Chagas' disease are limited by significant toxicity, including dermatotoxicity and digestive disorders. Furthermore, the effectiveness of current therapies is relatively modest, achieving parisotologic cures in only approximately 60% of acute patients, and is further limited by the apparent emergence of drug resistant T. cruzi.

The proteases of T. cruzi participate in the nutrition of the parasite at the expense of the host, but also appear to be involved in other aspects of the host-parasite relationship (see, e.g., Engel et al. (1998) J. Exp. Med. 188(4):725-734). For example, it has been suggested that the proteases may be involved in penetration of the parasite into the host cell, as well as in evasion of the immune response of the host.

One protease of T. cruzi that has been isolated and characterized is the cysteine protease cruzain, also referred to as cruzipain or gp 57/51. This 60 kDa protease exhibits sequence homology with a cysteine protease isolated from T. brucei and appears to be the major cysteine protease of T. cruzi. Among other uses for cruzain, it has been suggested that the enzyme is responsible for the intracellular digestion of human IgG bound to specific antigens at the parasite surface and taken up by endocytosis. Inhibition of cruzain has been reported to prevent growth and differentiation of T. cruzi in cell culture models of infection. Thus, there is a need for inhibitors of cruzain for use as therapeutic agents in treatment of Chagas' disease.

Therefore, it is an object herein to provide compounds, compositions and methods for modulating the activity of cruzain, particularly of inhibiting cruzain. It is a further object herein to provide compounds and compositions for treatment, prevention, or amelioration of one or more symptoms of Chagas' disease.

### SUMMARY OF THE INVENTION

Compounds and compositions useful as anti-parasitic agents are provided. The compounds and compositions are useful in the treatment, prevention, or amelioration of one or more symptoms of malaria or Chagas' disease. In particular, the compounds are active in assays that measure inhibition of the cysteine proteases falcipain or cruzain. The compositions contain compounds that are active in assays that measure inhibition of falcipain or cruzain. The compounds are acrylate, acrylamide, α-ketoamide and aldehyde derivatives of peptides, particularly dipeptides.

The compounds provided herein have formula I:

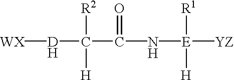

and pharmaceutically acceptable derivatives thereof, in which W is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, unsubstituted or substituted heteroaralkyl, unsubstituted or substituted alkylheteroaryl, unsubstituted or substituted heterocyclyl, or unsubstituted or substituted bicycloalkyl, bicycloalkenyl or bicycloalkynyl;

X is a direct link, —C(O)—, —OC(O)— or —S(O)ₙ— where n is an integer from 0 to 2;

D is nitrogen;

R² is selected from among H, alkyl, alkenyl, alkynyl, oxaalkyl, oxaalkenyl, oxaalkynyl, hydroxyalkyl, hydroxyalkenyl, hydroxyalkynyl, -alkylcarbonylalkyl, -alkenylcarbonylalkenyl, -alkynylcarbonylalkynyl, -alkylamine, -alkenylamine, -alkynylamine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylsulfurylalkyl, -alkenylsulfurylalkenyl, -alkynylsulfurylalkynyl, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, and unsubstituted or substituted heteroaralkyl;

E is carbon;

R¹ is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, or unsubstituted or substituted heteroaralkyl;

Y is —C(O)—, —A′H═CHC(O)— or —A′(O)C(O)NH— where A′ is carbon; and Z is G, J or L where G is hydrogen; and J and L are each independently selected from among H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, alkylamino, arylamino, dialkylamino, (alkyl)(aryl)amino, diarylamino, heteroarylamino, diheteroarylamino, (alkyl) (heteroaryl)amino, (aryl) (heteroaryl)amino, amino, heteroaryl or heterocyclyl.

In certain embodiments, the compounds are selected with the provisos that (i) if Y is —C(O)— or —A′(O)C(O)NH, then R¹ is not hydroxyalkyl, hydroxyalkenyl, hydroxyalkynyl, -alkylamino, -alkenylamine, -alkynylamine, alkylthiol, alkenylthiol or alkynylthiol; (ii) if X is —OC(O)—, then D is not attached ot oxygen; (iii) if Y is —C(O)—, then Z is G; (iv) if Y is —A′H═CHC(O)—, then Z is J; (v) if Y is —A′(O)C(O)NH—, then Z is L; and/or (vi) is Y is —A′H═CHC(O)— or —A′(O)C(O)NH—, then E is attached to A′.

In the above compounds, the alkyl, alkenyl and alkynyl groups contain from about 1 to about 12 carbon atoms. Preferred alkyl, alkenyl and alkynyl groups are lower alkyl, lower alkenyl and lower alkynyl groups, which, as defined herein, contain up to about 6 carbon atoms.

Also of interest are any pharmaceutically-acceptable derivatives, including salts, esters, acids, enol ethers and esters, bases, solvates, hydrates and prodrugs of the compounds described herein. Pharmaceutically-acceptable salts, include, but are not limited to, amine salts, such as but not limited to N,N′-dibenzylethylenediamine, chloroprocaine, choline, ammonia, diethanolamine and other hydroxyalkylamines, ethylenediamine, N-methylglucamine, procaine, N-benzylphenethylamine, 1-para-chlorobenzyl-2-pyrrolidin-1′-ylmethylbenzimidazole, diethylamine and other alkylamines, piperazine and tris(hydroxymethyl)aminomethane; alkali metal salts, such as but not limited to lithium, potassium and sodium; alkali earth metal salts, such as but not limited to barium, calcium and magnesium; transition metal salts, such as but not limited to zinc; and other metal salts, such as but not limited to sodium hydrogen phosphate and disodium phosphate; and also including, but not limited to, salts of mineral acids, such as but not limited to hydrochlorides and sulfates; and salts of organic acids, such as but not limited to acetates, lactates, malates, tartrates, citrates, ascorbates, succinates, butyrates, valerates and fumarates.

Pharmaceutical compositions formulated for administration by an appropriate route and means containing effective concentrations of one or more of the compounds provided herein, or pharmaceutically acceptable derivatives thereof, that deliver amounts effective for the treatment, prevention, or amelioration of one or more symptoms of parasitic infections, particularly malaria and Chagas' disease, are also provided. The effective amounts and concentrations are effective for ameliorating any of the symptoms of any of the disorders.

Methods for treatment, prevention, or amelioration of one or more symptoms of parasitic infections, particularly malaria or Chagas' disease, using one or more of the compounds provided herein, or pharmaceutically acceptable derivatives thereof, are provided.

Methods of modulating the activity of falcipain using the compounds and compositions provided herein are also provided. The compounds and compositions provided herein are active in assays that measure the activity of falcipain. Preferred are methods of inhibiting the activity of falcipain.

Methods of modulating the activity of cruzain using the compounds and compositions provided herein are also provided. The compounds and compositions provided herein are active in assays that measure the activity of cruzain. Preferred are methods of inhibiting the activity of cruzain.

Methods of inhibiting the development or growth of mammalian parasites, particularly malarial parasites or parasites that are the causative agent of Chagas' disease, more particularly Plasmodium falciparum, Trypanosoma cruzi or Trypanosoma brucei, are also provided.

In practicing the methods, effective amounts of the compounds, pharmaceutically acceptable derivatives thereof, or compositions containing therapeutically effective concentrations of the compounds, or pharmaceutically acceptable derivatives thereof, formulated for oral, intravenous, local or topical application for the treatment of parasitic infection, particularly malaria or Chagas' disease, are administered to an individual exhibiting the symptoms of these disorders. The amounts are effective to ameliorate or eliminate one or more symptoms of the disorders.

Articles of manufacture containing packaging material, a compound or composition, or pharmaceutically acceptable derivative thereof, provided herein, which is effective for inhibiting falcipain or cruzain or for treatment, prevention or amelioration of one or more symptoms of parasitic infections, particularly malaria or Chagas' disease, within the packaging material, and a label that indicates that the compound or composition, or pharmaceutically acceptable derivative thereof, is used for inhibiting falcipain or cruzain, or for treatment, prevention or amelioration of one or more symptoms of parasitic infection, particularly malaria or Chagas' disease, are provided.

### DETAILED DESCRIPTION OF PREFERRED EMBODIMENTS

A. Definitions

Unless defined otherwise, all technical and scientific terms used herein have the same meaning as is commonly understood by one of skill in the art to which this invention belongs. All patents, patent applications and publications referred to herein are incorporated by reference in their entirety.

As used herein, malaria refers to an acute and sometimes chronic infectious disease caused by or associated with parasitic infection, particularly infection with the protozoan parasites Plasmodium vivax, Plasmodium falciparum, Plasmodium malariae or Plasmodium ovale. The disease is characterized by the presence of the protozoan parasites within red blood cells. Of particular interest herein is malaria caused by or associated with P. falciparum infection.

As used herein, falcipain refers to a P. falciparum cysteine protease of the papain family. Falcipain is implicated in hemoglobin degradation in the parasitic food vacuole and in parasite development.

As used herein, Chagas' disease refers to a parasitic disease associated with or caused by infection with the protozoan parasite Trypanosoma cruzi.

As used herein, cruzain, also known as cruzipain or gp 57/51, refers to the major cysteine protease of T. cruzi. Cruzain is a 60 kDa high-mannose type glycoprotein.

As used herein, the IC₅₀ refers to a concentration of a particular test compound that achieves a 50% inhibition of a maximal response, such as inhibition of falcipain or cruzain.

As used herein, EC₅₀ refers to a concentration of a particular test compound that elicits a dose-dependent response at 50% of maximal expression of a particular response that is induced, provoked or potentiated by the particular test compound.

As used herein, pharmaceutically acceptable derivatives of a compound include salts, esters, enol ethers, enol esters, acids, bases, solvates, hydrates or prodrugs thereof that may be readily prepared by those of skill in this art using known methods for such derivatization and that produce compounds that may be administered to animals or humans without substantial toxic effects and that either are pharmaceutically active or are prodrugs. For example, acidic groups can be esterified or neutralized.

As used herein, treatment means any manner in which one or more of the symptoms of a condition, disorder or disease are ameliorated or otherwise beneficially altered. Treatment also encompasses any pharmaceutical use of the compositions herein, such as use as contraceptive agents.

As used herein, amelioration of the symptoms of a particular disorder by administration of a particular pharmaceutical composition refers to any lessening, whether permanent or temporary, lasting or transient that can be attributed to or associated with administration of the composition.

As used herein, biological activity refers to the in vivo activities of a compound or physiological responses that result upon in vivo administration of a compound, composition or other mixture. Biological activity, thus, encompasses therapeutic effects and pharmaceutical activity of such compounds, compositions and mixtures.

As used herein, a prodrug is a compound that, upon in vivo administration, is metabolized or otherwise converted to the biologically, pharmaceutically or therapeutically active form of the compound. To produce a prodrug, the pharmaceutically active compound is modified such that the active compound will be regenerated by metabolic processes. The prodrug may be designed to alter the metabolic stability or the transport characteristics of a drug, to mask side effects or toxicity, to improve the flavor of a drug or to alter other characteristics or properties of a drug. By virtue of knowledge of pharmacodynamic processes and drug metabolism in vivo, those of skill in this art, once a pharmaceutically active compound is known, can design prodrugs of the compound (see, e.g., Nogrady (1985) Medicinal Chemistry A Biochemical Approach, Oxford University Press, New York, pages 388-392).

It is to be understood that the compounds provided herein may contain chiral centers. Such chiral centers may be of either the (R) or (S) configuration, or may be a mixture thereof. Thus, the compounds provided herein may be enantiomerically pure, or be stereoisomeric or diastereomeric mixtures. In the case of amino acid residues, such residues may be of either the L- or D-form. The preferred configuration for naturally occurring amino acid residues is L. It is to be understood that the chiral centers of the compounds provided herein may undergo epimerization in vivo. As such, one of skill in the art will recognize that administration of a compound in its (R) form is equivalent, for compounds that undergo epimerization in vivo, to administration of the compound in its (S) form.

As used herein, substantially pure means sufficiently homogeneous to appear free of readily detectable impurities as determined by standard methods of analysis, such as thin layer chromatography (TLC), gel electrophoresis, high performance liquid chromatography (HPLC) and mass spectrometry (MS), used by those of skill in the art to assess such purity, or sufficiently pure such that further purification would not detectably alter the physical and chemical properties, such as enzymatic and biological activities, of the substance. Methods for purification of the compounds to produce substantially chemically pure compounds are known to those of skill in the art. A substantially chemically pure compound may, however, be a mixture of stereoisomers. In such instances, further purification might increase the specific activity of the compound.

As used herein, alkyl, alkenyl and alkynyl carbon chains, if not specified, contain from 1 to 20 carbons, preferably 1 to 1 6 carbons, and are straight or branched. Alkenyl carbon chains of from 2 to 20 carbons preferably contain 1 to 8 double bonds, and the alkenyl carbon chains of 1 to 1 6 carbons preferably contain 1 to 5 double bonds. Alkynyl carbon chains of from 2 to 20 carbons preferably contain 1 to 8 triple bonds, and the alkynyl carbon chains of 2 to 16 carbons preferably contain 1 to 5 triple bonds. Exemplary alkyl, alkenyl and alkynyl groups herein include, but are not limited to, methyl, ethyl, propyl, isopropyl, isobutyl, n-butyl, sec-butyl, tert-butyl, isopentyl, neopentyl, tert-penytyl and isohexyl. The alkyl, alkenyl and alkynyl groups, unless otherwise specified, may be optionally substituted, with one or more groups, preferably alkyl group substituents that may be the same or different. As used herein, lower alkyl, lower alkenyl, and lower alkynyl refer to carbon chains having less than about 6 carbons. As used herein, “alk(en)(yn)yl” refers to an alkyl group containing at least one double bond and at least one triple bond.

As used herein, an “alkyl group substituent” includes halo, haloalkyl, preferably halo lower alkyl, aryl, hydroxy, alkoxy, aryloxy, alkyloxy, alkylthio, arylthio, aralkyloxy, aralkylthio, carboxy alkoxycarbonyl, oxo and cycloalkyl.

As used herein, “aryl” refers to cyclic groups containing from 5 to 19 carbon atoms. Aryl groups include, but are not limited to groups, such as fluorenyl, substituted fluorenyl, phenyl, substituted phenyl, naphthyl and substituted naphthyl, in which the substituent is lower alkyl, halogen, or lower alkoxy.

As used herein, an “aryl group substituent” includes alkyl, cycloalkyl, cycloalkylalkyl, aryl, heteroaryl optionally substituted with 1 or more, preferably 1 to 3, substituents selected from halo, halo alkyl and alkyl, aralkyl, heteroaralkyl, alkenyl containing 1 to 2 double bonds, alkynyl containing 1 to 2 triple bonds, alk(en)(yn)yl groups, halo, pseudohalo, cyano, hydroxy, haloalkyl and polyhaloalkyl, preferably halo lower alkyl, especially trifluoromethyl, formyl, alkylcarbonyl, arylcarbonyl that is optionally substituted with 1 or more, preferably 1 to 3, substituents selected from halo, halo alkyl and alkyl, heteroarylcarbonyl, carboxy, alkoxycarbonyl, aryloxycarbonyl, aminocarbonyl, alkylaminocarbonyl, dialkylaminocarbonyl, arylaminocarbonyl, diarylaminocarbonyl, aralkylaminocarbonyl, alkoxy, aryloxy, perfluoroalkoxy, alkenyloxy, alkynyloxy, arylalkoxy, aminoalkyl, alkylaminoalkyl, dialkylaminoalkyl, arylaminoalkyl, amino, alkylamino, dialkylamino, arylamino, alkylarylamino, alkylcarbonylamino, arylcarbonylamino, azido, nitro, mercapto, alkylthio, arylthio, perfluoroalkylthio, thiocyano, isothiocyano, alkylsulfinyl, alkylsulfonyl, arylsulfinyl, arylsulfonyl, aminosulfonyl, alkylaminosulfonyl, dialkylaminosulfonyl and arylaminosulfonyl.

As used herein, “aralkyl” refers to an alkyl group in which one of the hydrogen atoms of the alkyl is replaced by an aryl group.

As used herein, “heteroaralkyl” refers to an alkyl group in which one of the hydrogen atoms of the alkyl is replaced by a heteroaryl group.

As used herein, “cycloalkyl” refers to a saturated mono- or multi-cyclic ring system, preferably of 3 to 10 carbon atoms, more preferably 3 to 6 carbon atoms; cycloalkenyl and cycloalkynyl refer to mono- or multicyclic ring systems that respectively include at least one double bond and at least one triple bond. Cycloalkenyl and cycloalkynyl groups may preferably contain 3 to 10 carbon atoms, with cycloalkenyl groups more preferably containing 4 to 7 carbon atoms and cycloalkynyl groups more preferably containing 8 to 1 0 carbon atoms. The ring systems of the cycloalkyl, cycloalkenyl and cycloalkynyl groups may be composed of one ring or two or more rings which may be joined together in a fused, bridged or spiro-connected fashion, and may be optionally substituted with one or more alkyl group substituents. “Cycloalk(en)(yn)yl” refers to a cylcoalkyl group containing at least one double bond and at least one triple bond.

As used herein, “heteroaryl” refers to a monocyclic or multicyclic ring system, preferably of about 5 to about 15 members where one or more, more preferably 1 to 3 of the atoms in the ring system is a heteroatom, that is, an element other than carbon, for example, nitrogen, oxygen and sulfur atoms. The heteroaryl may be optionally substituted with one or more, preferably 1 to 3, aryl group substituents. The heteroaryl group may be optionally fused to a benzene ring. Exemplary heteroaryl groups include, for example, furyl, imidazinyl, pyrrolidinyl, pyrimidinyl, tetrazolyl, thienyl, pyridyl, pyrrolyl, N-methylpyrrolyl, quinolinyl and isoquinolinyl, with pyridyl and quinolinyl being preferred.

As used herein, “heterocyclic” refers to a monocyclic or multicyclic ring system, preferably of 3 to 10 members, more preferably 4 to 7 members, even more preferably 5 to 6 members, where one or more, preferably 1 to 3 of the atoms in the ring system is a heteroatom, that is, an element other than carbon, for example, nitrogen, oxygen and sulfur atoms. The heterocycle may be optionally substituted with one or more, preferably 1 to 3 aryl group substituents. Preferred substituents of the heterocyclic group include hydroxy, amino, alkoxy containing 1 to 4 carbon atoms, halo lower alkyl, including trihalomethyl, such as trifluoromethyl, and halogen. As used herein, the term heterocycle may include reference to heteroaryl.

As used herein, the nomenclature alkyl, alkoxy, carbonyl, etc. are used as is generally understood by those of skill in this art. For example, as used herein alkyl refers to saturated carbon chains that contain one or more carbons; the chains may be straight or branched or include cyclic portions or be cyclic. As used herein, alicyclic refers to aryl groups that are cyclic.

Where the number of any given substituent is not specified (e.g., “haloalkyl”), there may be one or more substituents present. For example, “haloalkyl” may include one or more of the same or different halogens. As another example, “C₁₋₃alkoxyphenyl” may include one or more of the same or different alkoxy groups containing one, two or three carbons.

As used herein, “halogen” or “halide” refers to F, Cl, Br or I.

As used herein, pseudohalides are compounds that behave substantially similar to halides. Such compounds can be used in the same manner and treated in the same manner as halides (X⁻, in which X is a halogen, such as Cl or Br). Pseudohalides include, but are not limited to, cyanide, cyanate, thiocyanate, selenocyanate, trifluoromethoxy, trifluoromethyl and azide.

As used herein, “haloalkyl” refers to a lower alkyl radical in which one or more of the hydrogen atoms are replaced by halogen including, but not limited to, chloromethyl, trifluoromethyl, 1-chloro-2-fluoroethyl and the like.

As used herein, “haloalkoxy” refers to RO— in which R is a haloalkyl group.

As used herein, “sulfinyl” or “thionyl” refers to —S(O)—. As used herein, “sulfonyl” or “sulfuryl” refers to —S(O)₂—. As used herein, “sulfo” refers to —S(O)₃—.

As used herein, “carboxy” refers to a divalent radical, —OC(O)—.

As used herein, “aminocarbonyl” or “carbamoyl” refers to —C(O)NH₂.

As used herein, “alkylaminocarbonyl” refers to —C(O)NHR in which R is hydrogen or alkyl, preferably lower alkyl. As used herein “dialkyl-aminocarbonyl” as used herein refers to —C(O)NR′R in which R′ and R are independently selected from hydrogen or alkyl, preferably lower alkyl; “carboxamide” refers to groups of formula —NR′COR.

As used herein, “diarylaminocarbonyl” refers to —C(O)NRR′ in which R and R′ are independently selected from aryl, preferably lower aryl, more preferably phenyl.

As used herein, “aralkylaminocarbonyl” refers to —C(O)NRR′ in which one of R and R′ is aryl, preferably lower aryl, more preferably phenyl, and the other of R and R′ is alkyl, preferably lower alkyl.

As used herein, “arylaminocarbonyl” refers to —C(O)NHR in which R is aryl, preferably lower aryl, more preferably phenyl.

As used herein, “alkoxycarbonyl” refers to —C(O)OR in which R is alkyl, preferably lower alkyl.

As used herein, “aryloxycarbonyl” refers to —C(O)OR in which R is aryl, preferably lower aryl, more preferably phenyl.

As used herein, “alkoxy” and “alkylthio” refer to RO— and RS—, in which R is alkyl, preferably lower alkyl.

As used herein, “aryloxy” and “arylthio” refer to RO— and RS—, in which R is aryl, preferably lower aryl, more preferably phenyl.

As used herein, “alkylene” refers to a straight, branched or cyclic, preferably straight or branched, bivalent aliphatic hydrocarbon group, preferably having from 1 to about 20 carbon atoms, more preferably 1 to 12 carbons, even more preferably lower alkylene. The alkylene group is optionally substituted with one or more “alkyl group substituents.” There may be optionally inserted along the alkylene group one or more oxygen, sulphur or substituted or unsubstituted nitrogen atoms, where the nitrogen substituent is alkyl as previously described. Exemplary alkylene groups include methylene (—CH₂—), ethylene (—CH₂CH₂—), propylene (—(CH₂)₃—), cyclohexylene (—C₆H₁₀—), methylenedioxy (—O—CH₂—O—) and ethylenedioxy (—O—(CH₂)₂—O—). The term “lower alkylene” refers to alkylene groups having 1 to 6 carbons. Preferred alkylene groups are lower alkylene, with alkylene of 1 to 3 carbon atoms being particularly preferred.

As used herein, “alkenylene” refers to a straight, branched or cyclic, preferably straight or branched, bivalent aliphatic hydrocarbon group, preferably having from 2 to about 20 carbon atoms and at least one double bond, more preferably 1 to 1 2 carbons, even more preferably lower alkenylene. The alkenylene group is optionally substituted with one or more “alkyl group substituents.” There may be optionally inserted along the alkenylene group one or more oxygen, sulphur or substituted or unsubstituted nitrogen atoms, where the nitrogen substituent is alkyl as previously described. Exemplary alkenylene groups include —CH═CH—CH═CH— and —CH═CH—CH₂—. The term “lower alkenylene” refers to alkenylene groups having 2 to 6 carbons. Preferred alkenylene groups are lower alkenylene, with alkenylene of 3 to 4 carbon atoms being particularly preferred.

As used herein, “alkynylene” refers to a straight, branched or cyclic, preferably straight or branched, bivalent aliphatic hydrocarbon group, preferably having from 2 to about 20 carbon atoms and at least one triple bond, more preferably 1 to 12 carbons, even more preferably lower alkynylene. The alkynylene group is optionally substituted with one or more “alkyl group substituents.” There may be optionally inserted along the alkynylene group one or more oxygen, sulphur or substituted or unsubstituted nitrogen atoms, where the nitrogen substituent is alkyl as previously described. Exemplary alkynylene groups include —C≡C—C≡C—, —C≡C— and —C≡C—CH₂—. The term “lower alkynylene” refers to alkynylene groups having 2 to 6 carbons. Preferred alkynylene groups are lower alkynylene, with alkynylene of 3 to 4 carbon atoms being particularly preferred.

As used herein, “alk(en)(yn)ylene” refers to a straight, branched or cyclic, preferably straight or branched, bivalent aliphatic hydrocarbon group, preferably having from 2 to about 20 carbon atoms and at least one triple bond, and at least one double bond; more preferably 1 to 12 carbons, even more preferably lower alk(en)(yn)ylene. The alk(en)(yn)ylene group is optionally substituted with one or more “alkyl group substituents.” There may be optionally inserted along the alkynylene group one or more oxygen, sulphur or substituted or unsubstituted nitrogen atoms, where the nitrogen substituent is alkyl as previously described. Exemplary alk(en)(yn)ylene groups include —C═C—(CH₂)ₙ—C≡C—, where n is 1 or 2. The term “lower alk(en)(yn)ylene” refers to alk(en)(yn)ylene groups having up to 6 carbons. Preferred alk(en)(yn)ylene groups are lower alk(en)(yn)ylene, with alk(en)(yn)ylene of 4 carbon atoms being particularly preferred.

As used herein, “arylene” refers to a monocyclic or polycyclic, preferably monocyclic, bivalent aromatic group, preferably having from 5 to about 20 carbon atoms and at least one aromatic ring, more preferably 5 to 12 carbons, even more preferably lower arylene. The arylene group is optionally substituted with one or more “alkyl group substituents.” There may be optionally inserted around the arylene group one or more oxygen, sulphur or substituted or unsubstituted nitrogen atoms, where the nitrogen substituent is alkyl as previously described. Exemplary arylene groups include 1,2-, 1,3- and 1,4-phenylene. The term “lower arylene” refers to arylene groups having 5 or 6 carbons. Preferred arylene groups are lower arylene.

As used herein, “heteroarylene” refers to a bivalent monocyclic or multicyclic ring system, preferably of about 5 to about 1 5 members where one or more, more preferably 1 to 3 of the atoms in the ring system is a heteroatom, that is, an element other than carbon, for example, nitrogen, oxygen and sulfur atoms. The heteroarylene group may be optionally substituted with one or more, preferably 1 to 3, aryl group substituents.

As used herein, “alkylidene” refers to a bivalent group, such as ═CR′R″, which is attached to one atom of another group, forming a double bond. Exemplary alkylidene groups are methylidene (═CH₂) and ethylidene (═CHCH₃). As used herein, “aralkylidene” refers to an alkylidene group in which either R′ or R″ is and aryl group.

As used herein, “amido” refers to the bivalent group —C(O)NH—. “Thioamido” refers to the bivalent group —C(S)NH—. “Oxyamido” refers to the bivalent group —OC(O)NH—. “Thiaamido” refers to the bivalent group —SC(O)NH—. “Dithiaamido” refers to the bivalent group —SC(S)NH—. “Ureido” refers to the bivalent group —HNC(O)NH—. “Thioureido” refers to the bivalent group —HNC(S)NH—.

As used herein, “semicarbazide” refers to —NHC(O)NHNH—. “Carbazate” refers to the bivalent group —OC(O)NHNH—. “Isothiocarbazate” refers to the bivalent group —SC(O)NHNH—. “Thiocarbazate” refers to the bivalent group —OC(S)NHNH—. “Sulfonylhydrazide” refers to the group —SO₂NHNH—. “Hydrazide” refers to the bivalent group —C(O)NHNH—. “Azo” refers to the bivalent group —N═N—. “Hydrazinyl” refers to the bivalent group —NH—NH—.

As used herein, the term “amino acid” refers to α-amino acids which are racemic, or of either the D- or L-configuration. The designation “d” preceding an amino acid designation (e.g., dAla, dSer, dVal, etc.) refers to the D-isomer of the amino acid. The designation “dl” preceding an amino acid designation (e.g., dlPip) refers to a mixture of the L- and D-isomers of the amino acid.

As used herein, when any particular group, such as phenyl or pyridyl, is specified, this means that the group is unsubstituted or is substituted. Preferred substituents where not specified are halo, halo lower alkyl, and lower alkyl.

As used herein, the abbreviations for any protective groups, amino acids and other compounds, are, unless indicated otherwise, in accord with their common usage, recognized abbreviations, or the IUPAC-IUB Commission on Biochemical Nomenclature (see, Biochem. 1972, 11, 942).

B. Compounds Useful as Falcipain or Cruzain Inhibitors

Compounds and compositions useful as falcipain or cruzain inhibitors are provided. The compositions contain compounds that are active in assays that measure falcipain or cruzain activity. The compounds and compositions provided herein are thus useful in treatment, prevention, or amelioration of one or more symptoms of disease states in which falcipain or cruzain are implicated, particularly parasitic infections such as malaria and Chagas' disease. In preferred embodiments, the compounds are α-ketoamide, acrylate, acrylamide and aldehyde derivatives of peptides, preferably dipeptides.

In all embodiments described herein, the compounds for use in the compositions and methods provided herein are α-ketoamide, acrylate, acrylamide and aldehyde derivatives of dipeptides of formula I in which R¹ is preferably an unsubstituted or substituted aryl, aralkyl, heteroaryl or heteroaralkyl group, more preferably an unsubstituted or substituted aryl or aralkyl group.

In one embodiment, the compounds for use in the compositions and methods have formula I:

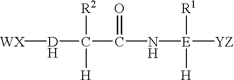

where W is hydrogen, alkyl, alkenyl, alkynyl, aralkyl, heteroaralkyl, aryl, heteroaryl, bicyclic alkyl or heterocyclyl; X is a direct link, —C(O)—, —OC(O)— or —SOₙ— where n is an integer from 0 to 2, preferably 2; D is nitrogen; R² is alkylalkenyl, alkynyl, aralkyl, heteroaralkyl, -alkylsulfurylalkyl, -alkenylsulfurylalkenyl or -alkynylsulfurylalkynyl; E is carbon; R¹ is selected from among aryl, heteroaryl, aralkyl and heteroaralkyl; Y is —C(O)—, —A′H═CHC(O)— or —A′(O)C(O)NH— where A′ is carbon; and Z is G, J or L where G is hydrogen; J is -alkylcarbamoyl, -alkenylcarbamoyl, -alkynylcarbamoyl, -alkylamide, -alkenylamide, -alkynylamide, substituted or unsubstituted aryl, substituted or unsubstituted aralkyl, substituted or unsubstituted heteroaryl, or substituted or unsubstituted heteroaralkyl; and L is oxaalkyl, oxaalkenyl, oxaalkynyl, alkylamino, arylamino, dialkylamino, (alkyl)(aryl)amino, diarylamino, heteroarylamino, diheteroarylamino, (alkyl)(heteroaryl)amino, (aryl)(heteroaryl)amino, amino, heteroaryl or heterocyclyl.

In certain embodiments herein, the compounds are of formula I in which W is hydrogen, C₁₋₄alkyl, benzyl, phenyl, camphoryl, C₁₋₄alkylpiperazinyl or morpholino; X is —C(O)—, —OC(O)— or —SO₂—; D is nitrogen; R² is the side chain of leucine (isobutyl) or phenylalanine (benzyl) or is —CH₂CH₂SO₂CH₃; E is carbon; R¹ is the side chain of tyrosine (4-hydroxybenzyl), phenylalanine (benzyl), homophenylalanine (2-phenyleth-1-yl) or 4-methoxyphenylalanine (4-methoxybenzyl); Y is —C(O)—, —A′H═CHC(O)—, or —A′(O)C(O)NH— where A′ is carbon; and Z is G, J or L, in which G is hydrogen; J is —CH(CH₂Ph)(CONH₂), —CH₂CH₂—(2-pyridyl), —CH₂CH₂Ph, —CH₂CHPh₂, —CH₂CH₂-(1-methyl-3-indolyl) or —CH₂CH₂-(1-benzyl-3-indolyl); and L is methoxy or 1-indolinyl.

In the above embodiments, the compounds are preferably selected with the provisos that (i) if X is —C(O)—; then D is not attached to oxygen; (ii) if Y is —C(O)—, then Z is G; (iii) if Y is —A′H═CHC(O)—, then Z is G or J; (iv) if Y is —A′(O)C(O)NH—, then Z is G or L; and (v) if Y is —A′H═CHC(O)— or —A′(O)C(O)NH—, then E is attached to A′.

1. Dipeptide Aldehyde Derivatives

In certain embodiments herein, Y is —C(O)—, Z is G and the compounds of formula I are dipeptide aldehyde derivatives that have formula II:

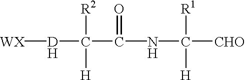

where W is hydrogen, alkyl, alkenyl, alkynyl, aralkyl, heteroaralkyl, aryl, heteroaryl, bicyclic alkyl or heterocyclyl; X is a direct link, —C(O)—, —OC(O)— or —SOₙ— where n is an integer from 0 to 2, preferably 2; D is nitrogen; R² is alkyl, alkylalkenyl, alkynyl, cycloalkylalkyl, aralkyl, heteroaralkyl, -alkylsulfurylalkyl, -alkenylsulfurylalkenyl or -alkynylsulfurylalkynyl; and R¹ is selected from among aryl, heteroaryl, aralkyl and heteroaralkyl. In certain embodiments, R¹ is preferably selected from aralkyl and heteroaralkyl where the alkyl portion contains from about 2 to about 6 carbons.

In preferred embodiments, the compounds are of formula 11 where W is hydrogen, C₁₋₄alkyl, benzyl, phenyl, camphoryl, C₁₋₄alkylpiperazinyl or morpholino; X is a direct link, —C(O)—, —OC(O)— or —SO₂—; D is nitrogen; R² is the side chain of leucine (isobutyl) or phenylalanine (benzyl) or is —CH₂CH₂SO₂CH₃; and R¹ is the side chain of tyrosine (4-hydroxybenzyl), phenylalanine (benzyl), homophenylalanine (2-phenyleth-1-yl) or 4-methoxyphenylalanine (4-methoxybenzyl).

In certain embodiments, the compounds of formula II are selected as described above with the proviso that if X is —C(O)—; then D is not attached to oxygen.

In more preferred embodiments, the compounds are of formula II where W is benzyl, phenyl, 4-methylpiperazinyl or morpholino, preferably 4-methylpiperazinyl or morpholino, more preferably morpholino; X is a direct link, —C(O)—, —OC(O)— or —SO₂—, preferably —C(O)— or —SO₂—, more preferably —C(O)—; D is nitrogen; R² is the side chain of leucine (isobutyl) or phenylalanine (benzyl); and R¹ is the side chain of tyrosine (4-hydroxybenzyl) or homophenylalanine (2-phenyleth-1-yl), preferably homophenylalanine (2-phenyleth-1-yl).

Presently preferred compounds of formula II include N-(N-(4-methylpiperazinylcarbonyl)leucyl)tyrosinal, (S)-2-(N-(4-methylpiperazinylcarbonyl)leucyl)amino-4-phenylbutanal, N-(N-(morpholinocarbonyl)-leucyl)tyrosinal, N-(N-(benzyloxycarbonyl)leucyl)tyrosinal, (S)-2-(N-(phenylsulfonyl)leucyl)amino-4-phenylbutanal, N-(N-(phenylsulfonyl)-leucyl)tyrosinal, (S)-2-(N-(morpholinocarbonyl)phenylalanyl)amino-4-phenylbutanal, N-(N-(benzyloxycarbonyl)phenylalanyl)tyrosinal, (S)-2-(N-(benzyloxycarbonyl)phenylalanyl)amino-4-phenylbutanal, (S)-2-(N-(phenylsulfonyl)phenylalanyl)amino-4-phenylbutanal, N-(N-(morpholinocarbonyl)-phenylalanyl)tyrosinal, N-(N-(4-methylpiperazinylcarbonyl)phenylalanyl)-tyrosinal, (S)-2-(N-(4-methylpiperazinylcarbonyl)phenylalanyl)amino-4-phenylbutanal, N-(N-(phenylsulfonyl)phenylalanyl)tyrosinal, (S)-2-(N-(benzyloxycarbonyl)leucyl)amino-4-phenylbutanal and (S)-2-(N-(morpholinocarbonyl)leucyl)amino-4-phenylbutanal.

More preferred compounds herein include N-(N-(4-methylpiperazinylcarbonyl)leucyl)tyrosinal, (S)-2-(N-(4-methylpiperazinylcarbonyl)leucyl)amino-4-phenylbutanal, N-(N-(morpholinocarbonyl)-leucyl)tyrosinal, (S)-2-(N-(phenylsulfonyl)leucyl)amino-4-phenylbutanal, N-(N-(phenylsulfonyl)leucyl)tyrosinal, (S)-2-(N-(morpholinocarbonyl)phenylalanyl)amino-4-phenylbutanal, (S)-2-(N-(phenylsulfonyl)phenylalanyl)-amino-4-phenylbutanal, N-(N-(morpholinocarbonyl)phenylalanyl)tyrosinal, N-(N-(4-methylpiperazinylcarbonyl)phenylalanyl)tyrosinal, (S)-2-(N-(4-methylpiperazinylcarbonyl)phenylalanyl)amino-4-phenylbutanal, N-(N-(phenylsulfonyl)phenylalanyl)tyrosinal, (S)-2-(N-(benzyloxycarbonyl)-leucyl)amino-4-phenylbutanal and (S)-2-(N-(morpholinocarbonyl)leucyl)-amino-4-phenylbutanal.

In certain embodiments, the preferred compounds herein include (S)-2-(N-(4-methylpiperazinylcarbonyl)leucyl)amino-4-phenylbutanal, (S)-2-(N-(phenylsulfonyl)leucyl)amino-4-phenylbutanal, (S)-2-(N-(morpholino-carbonyl)phenylalaninyl)amino-4-phenylbutanal, (S)-2-(N-(phenylsulfonyl)-phenylalaninyl)amino-4-phenylbutanal, (S)-2-(N-(4-methylpiperazinyl-carbonyl)phenylalaninyl)amino-4-phenylbutanal and (S)-2-(N-(morpholinocarbonyl)leucyl)amino-4-phenylbutanal.

In certain embodiments herein, the compound is of formula II with the proviso that the compound is not N-(N-(benzyloxycarbonyl)leucyl)-tyrosinal, N-(N-(benzyloxycarbonyl)phenylalanyl)tyrosinal or (S)-2-(N-(benzyloxycarbonyl)phenylalanyl)amino-4-phenylbutanal.

In other embodiments, the compounds are of formula II in which D is nitrogen; R² is alkyl, alkenyl, alkynyl, aralkyl, heteroaralkyl, -alkylsulfurylalkyl, -alkenylsulfurylalkenyl or -alkynylsulfurylalkynyl, preferably isobutyl, benzyl or —CH₂CH₂SO₂CH₃; and

R¹, W and X are selected from (i), (ii) or (iii) as follows:

(i) R¹ is aralkyl or heteroaralkyl, with the proviso that R¹ is not 3-indolylmethyl;

W is heteroaryl or heterocyclyl, preferably 4-methylpiperazin-yl or morpholino; and

X is —C(O)—; or

(ii) R¹ is aralkyl or heteroalkaryl;

W is alkyl, alkenyl, alkynyl, aralkyl, heteroaralkyl, aryl, heteroaryl, bicyclic alkyl or heterocyclyl, preferably aryl, heteroaryl or heterocyclyl, more preferably phenyl; and

X is —SOₙ— where n is an integer from 0 to 2;

with the provisos that (i) if X is SO₂, then R¹ is not subsituted or unsubstituted benzyl or CH₂heteroaryl; and (ii) if R¹ is —CH₂-(para-hydroxy)phenyl or —CH₂-(para-isopropoxy)-phenyl, then W is not naphthyl; or

(iii) R¹ is aralkyl or heteroalkaryl;

W is alkyl, alkenyl, alkynyl, aralkyl, heteroaralkyl, aryl, heteroaryl, bicyclic alkyl or heterocyclyl, preferably alkyl, aralkyl, aryl or bicyclic alkyl, more preferably benzyl; and

X is —OC(O)—;

with the provisos that (i) the alkyl portion of R¹ has 2 to 6 carbons; and (ii) if the alkyl portion of R¹ is ethylene, then R² is not isopropyl or benzyl.

In these embodiments, the alkyl portion of R¹ preferably has from about 2 to about 6 carbons. In other embodiments, R¹ is 4-hydroxy-benzyl or 2-phenyl-1-ethyl.

2. Dipeptide α-ketoamide Derivatives

In certain embodiments herein, Y is —A′(O)C(O)NH— where A′ is carbon, Z is J and the compounds of formula I are dipeptide a-ketoamide derivatives of formula III:

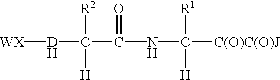

in which W is hydrogen, alkyl, alkenyl, alkynyl, aralkyl, heteroaralkyl, aryl, heteroaryl, bicyclic alkyl or heterocyclyl; X is a direct link, —C(O)—, —OC(O)—or —SOₙ— where n is an integer from 0 to 2, preferably 2; D is nitrogen; R² is alkylalkenyl, alkynyl, aralkyl, heteroaralkyl, -alkylsulfurylalkyl, -alkenylsulfurylalkenyl or -alkynylsulfurylalkynyl; R¹ is selected from among aryl, heteroaryl, aralkyl and heteroaralkyl; and J is -alkylcarbamoyl, -alkenylcarbamoyl, -alkynylcarbamoyl, -alkylamide, -alkenylamide, -alkynylamide, substituted or unsubstituted aryl, substituted or unsubstituted aralkyl, substituted or unsubstituted heteroaryl, or substituted or unsubstituted heteroaralkyl.

In certain embodiments herein, the compounds are of formula III in which R¹ is selected from among (i), (ii) or (iii) as follows:

(i) aryl or heteroaryl;

(ii) aralkyl where (a) the alkyl portion has one carbon atom and the aryl portion is substituted with at least one non-hydrogen substituent, or (b) the alkyl portion has at least two carbon atoms; or

(iii) heteroaralkyl.

In preferred embodiments, the compounds are of formula III where W is hydrogen, C₁₋₄alkyl, benzyl, phenyl, camphoryl, C-₁₋₄alkylpiperazinyl or morpholino; X is a direct link, —C(O)—, —OC(O)— or —SO₂—; D is nitrogen; R² is the side chain of leucine (isobutyl) or phenylalanine (benzyl) or is —CH₂CH₂SO₂CH₃; R¹ is the side chain of tyrosine (4-hydroxybenzyl), phenylalanine (benzyl), homophenylalanine (2-phenyleth-1-yl) or 4-methoxyphenylalanine (4-methoxybenzyl); and J is —CH(CH₂Ph)(CONH₂), —CH₂CH₂-(2-pyridyl), —CH₂CH₂Ph, —CH₂CHPh₂, —CH₂CH₂-(1-methyl-3-indolyl) or —CH₂CH₂-(1-benzyl-3-indolyl).

In certain embodiments, the compounds of formula III are selected as described above with the proviso that if X is —C(O)—; then D is not attached to oxygen.

In more preferred embodiments, the compounds are of formula III where W is benzyl, phenyl or morpholino; X is a direct link, —C(O)—, —OC(O)— or —SO₂—; D is nitrogen; R² is the side chain of leucine (isobutyl) or phenylalanine (benzyl); R¹ is the side chain of homophenylalanine (2-phenyleth-1-yl) or 4-methoxyphenylalanine (4-methoxybenzyl); and J is —CH(CH₂Ph)(CON H₂), —CH₂CH₂-(2-pyridyl), —CH₂CH₂Ph, —CH₂CHPh₂, —CH₂CH₂-(1-methyl-3-indolyl) or —CH₂CH₂-(1-benzyl-3-indolyl).

Preferred compounds of formula III herein include N-(2-phenyl-1-carbamoyl-1-ethyl)-3-((N-benzyloxycarbonyl)phenylalaninyl)-2-oxo-5-phenylpentanamide, N-(2-(2-pyridyl)-1-ethyl)-3-((N-benzyloxycarbonyl)-phenylalaninyl)-2-oxo-4-(4-methoxyphenyl)butanamide, N-(2-phenyl-1-ethyl)-3-((N-benzyloxycarbonyl)phenylalaninyl)-2-oxo-4-(4-methoxyphenyl)butanamide, N-(2-(2-pyridyl)-1-ethyl)-3-((N-benzyloxycarbonyl)-leucyl)-2-oxo-4-(4-methoxyphenyl)butanamide, N-(2,2-diphenyl-1-ethyl)-3-((N-benzyloxycarbonyl)phenylalaninyl)-2-oxo-4-(4-methoxyphenyl)butanamide, N-(2-(1-methyl-3-indolyl)-1-ethyl)-3-((N-benzyloxycarbonyl)leucyl)-2-oxo-4-(4-methoxyphenyl)butanamide, N-(2-(1-benzyl-3-indolyl)-1-ethyl)-3-((N-benzyloxycarbonyl)leucyl)-2-oxo-4-(4-methoxyphenyl)butanamide, N-(2-phenyl-1-carbamoyl-1-ethyl)-3-((N-morpholinocarbonyl)leucyl)-2-oxo-5-phenylpentanamide, N-(2-phenyl-1-ethyl)-3-((N-benzyloxycarbonyl)-leucyl)-2-oxo-4-(4-methoxyphenyl)butanamide, N-(2-phenyl-1-ethyl)-3-((N-benzyloxycarbonyl)phenylalaninyl)-2-oxo-4-(4-methoxyphenyl)butanamide, N-(2-phenyl-1-ethyl)-3-((N-phenylsulfonyl)leucyl)-2-oxo-4-(4-methoxyphenyl)butanamide and N-(2-phenyl-1-ethyl)-3-((N-phenylsulfonyl)phenylalaninyl)-2-oxo-4-(4-methoxyphenyl)butanamide.

3. Dipeptide Acrylamide and Acrylate Derivatives

In certain embodiments herein, Y is —A′H =CHC(O)—where A′ is carbon, Z is L, and the compounds of formula I are dipeptide acrylamide and acrylate derivatives of formula IV:

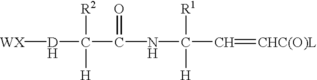

in which W is hydrogen, alkyl, alkenyl, alkynyl, aralkyl, heteroaralkyl, aryl, heteroaryl, bicyclic alkyl or heterocyclyl; X is a direct link, —C(O)—, —OC(O)— or —SOₙ— where n is an integer from 0 to 2, preferably 2; D is nitrogen; R² is alkylalkenyl, alkynyl, aralkyl, heteroaralkyl, -alkylsulfurylalkyl, -alkenylsulfurylalkenyl or -alkynylsulfurylalkynyl; R¹ is selected from among aryl, heteroaryl, aralkyl and heteroaralkyl; and L is oxaalkyl, oxaalkenyl, oxaalkynyl, alkylamino, arylamino, dialkylamino, (alkyl)(aryl)amino, diarylamino, heteroarylamino, diheteroarylamino, (alkyl)(heteroaryl)amino, (aryl)(heteroaryl)amino, amino, heteroaryl or heterocyclyl, preferably heteroaryl or heterocyclyl.

In certain embodiments, the compounds are of formula IV in which R¹ is selected from among (i) or (ii) as follows:

(i) aryl, heteroaryl or heteroaralkyl; or

(ii) aralkyl where (a) the alkyl portion has at least two carbon atoms, or (b) the alkyl portion has one carbon atom and R² is not benzyl, 3-indolylmethyl or isopropyl; and

In preferred embodiments, the compounds are of formula IV where W is hydrogen, C₁₄alkyl, benzyl, phenyl, camphoryl, C₁₋₄alkylpiperazinyl or morpholino; X is a direct link, —C(O)—, —OC(O)— or —SO₂—; D is nitrogen; R² is the side chain of leucine (isobutyl) or phenylalanine (benzyl) or is —CH₂CH₂SO₂CH₃; R¹ is the side chain of tyrosine (4-hydroxybenzyl), phenylalanine (benzyl), homophenylalanine (2-phenyleth-1-yl) or 4-methoxyphenylalanine (4-methoxybenzyl); and L is oxaalkyl or heterocyclyl, preferably heterocyclyl.

In more preferred embodiments, the compounds are of formula IV where W is C₁₋₄alkyl or benzyl, preferably tert-butyl or benzyl; X is —OC(O)—or —SO₂—; D is nitrogen; R² is the side chain of leucine (isobutyl) or phenylalanine (benzyl) or is —CH₂CH₂SO₂CH₃; R¹ is the side chain of phenylalanine (benzyl) or homophenylalanine (2-phenyleth-1-yl); and L is methoxy or 1-indolinyl, preferably 1-indolinyl.

Preferred compounds of formula IV herein include methyl (E)-4-((N-(benzyloxycarbonyl)phenylalaninyl)amino)-5-phenyl-2-pentenoate, methyl (E)-4-((N-(benzyloxycarbonyl)phenylalaninyl)amino)-6-phenyl-2-hexenoate, N-(1-indolinyl)-(E)-4-((N-(tert-butoxycarbonyl)phenylalaninyl)amino)-6-phenyl-2-hexenamide, N-(1-indolinyl)-(E)-4-((N-(tert-butoxycarbonyl)-leucyl)amino)-6-phenyl-2-hexenamide, N-(1-indolinyl)-(E)-4-((N-tert-butoxycarbonyl-S,S-dioxomethioninyl)amino)-6-phenyl-2-hexenoate, N-(1-indolinyl)-(E)-4-((N-(benzylsulfonyl)phenylalaninyl)amino)-6-phenyl-2-hexenamide and N-(1-indolinyl)-(E)-4-((N-(benzylsulfonyl)leucyl)amino)-6-phenyl-2-hexenamide.

C. Preparation of the Compounds

The preparation of the above compounds is described below. Any such compound or similar compound may be synthesized according to a method discussed in general below or by only minor modification of the methods by selecting appropriate starting materials.

As used herein the symbols and conventions used in these processes, schemes and examples are consistent with those used in the contemporary scientific literature, for example, the Journal of the American Chemical Society or the Journal of Biological Chemistry. Standard three-letter abbreviations are generally used to designate amino acid residues, which are assumed to be in the L-configuration unless otherwise noted. Unless otherwise noted, all starting materials were obtained from commercial suppliers and used without further purification. Specifically, the following abbreviations may be used in the examples and throughout the specification: g (grams); mg (milligrams); L (liters); mL (milliliters); μL (microliters); psi (pounds per square inch); M (molar); mmol (millimolar); i. v. (intravenous); Hz (Hertz); MHz (megahertz); mol (moles); mmol (millimoles); RT (room temperature); min (minutes); h (hours); mp (melting point); TLC (thin layer chromatography); HPLC (high pressure liquid chromatography); Rₜ(retention time); RP (reverse phase); MeOH (methanol); i-PrOH (isopropanol); Et₃N (triethylamine); TFA (trifluoroacetic acid); THF (tetrahydrofuran); DMSO (dimethylsulfoxide); EtOAc (ethyl acetate); DCM (dichloromethane); 4-NMM (N-methylmorpholine); LAH (lithium aluminum hydride); Dibal-H (diisobutylaluminum hydride); DCE (dichloroethane); DMF (N,N-dimethylformamide); AcOH (acetic acid); HOAt (1-hydroxy-7-azabenzotriazole); EDC (ethylcarbodiimide hydrochloride); Boc (tert-butyloxycarbonyl); FMOC (9-fluorenylmethoxycarbonyl); Z (benzyloxycarbonyl); Ac (acetyl); and atm (atmosphere). All references to ether are to diethyl ether; brine refers to a saturated aqueous solution of NaCl; and Rochelle salt refers to sodium potassium tartrate. Unless otherwise indicated, all temperatures are expressed in ° C. (degrees Centigrade). All reactions conducted under an inert atmosphere at room temperature unless otherwise noted.

¹H NMR spectra were recorded on a Varian Unity Inova-400 instrument. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), br (broad).

Low-resolution mass spectra (MS) were recorded on a Perkin Elmer SCIE API1 spectrometer. All reactions were monitored by thin-layer chromatography on 0.25 mm E. Merck silica gel plates (60F-254), visualized with UV light, 5% ethanolic phosphomolybdic acid. Flash column chromatography was performed on silica gel (230-400 mesh, Merck).

In scheme(s) described below, it is well understood that protecting groups for sensitive or reactive groups are employed where necessary in accordance with general principles of chemistry. Protecting groups are manipulated according to standard methods of organic synthesis (T. W. Green and P. G. M. Wuts (1991) Protecting Groups in Organic Synthesis, John Wiley & Sons). These groups are removed at a convenient stage of the compound synthesis using methods that are readily apparent to those skilled in the art.

Those skilled in the art will recognize that when a stereocenter(s) exists in compounds of formula I, the single enantiomer may be obtained by stereospecific synthesis or by resolution of the final product or any convenient intermediate. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994).

Compounds of formula I may be prepared according to the synthetic sequence shown in scheme 1. Unless otherwise specified, variables R¹, R², W, X, D and L in scheme I have values of formula I as described hereinabove. An appropriate N-protected amino acid (a) (P=an amino acid protecting group) was transformed into aldehyde (c) by reduction of the corresponding Weinreb amide (b). The same aldehyde was also obtained in one step by reduction of the corresponding methyl or ethyl ester derivatives (d) with diisobutylaluminum hydride in toluene at −78° C. The crude aldehyde thus obtained was subjected to a Wittig reaction employing methyl (triphenylphosphoranylidene)acetate (R⁴⁰=Me) to provide olefin (e) after purification on silica gel. Methyl ester was carefully hydrolyzed under basic conditions to afford (f) with a minimal amount of racemization. The carboxylic acid intermediate was condensed with the appropriate amine or alcohol group to obtain the desired Michael acceptor (g). Removal of the protecting group followed by coupling with the desired acid led to the synthesis of the desired target (h).

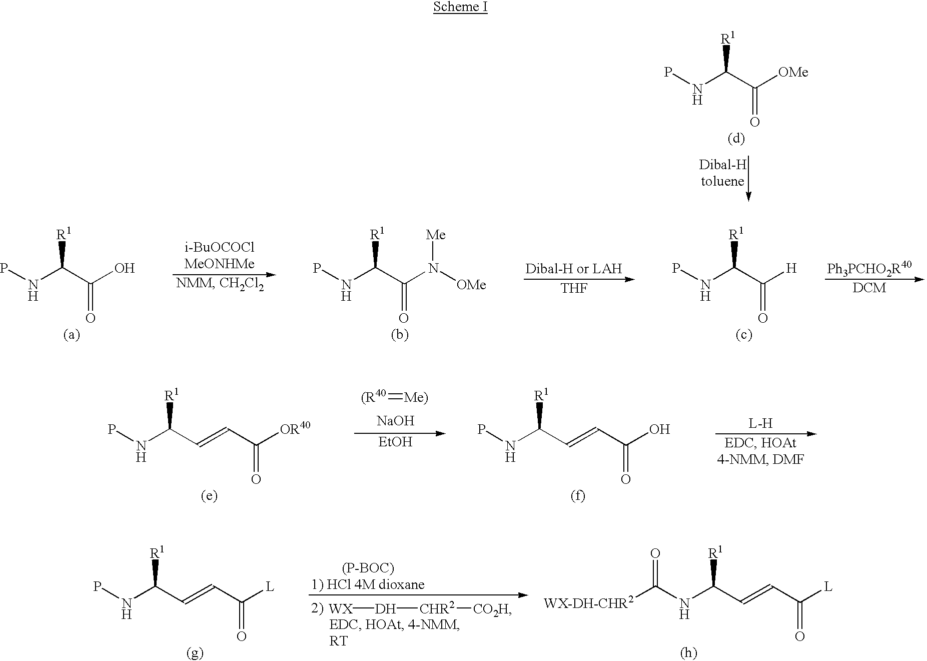

D. Formulation of Pharmaceutical Compositions

The pharmaceutical compositions provided herein contain therapeutically effective amounts of one or more falcipain or cruzain inhibitors of formula I that are useful in the prevention, treatment, or amelioration of one or more of the symptoms of parasitic infections, particularly malaria or Chagas' disease. The compositions contain one or more acrylamide, acrylate, α-ketoamide or aldehyde derivatives of peptides, particularly dipeptides. Preferred compounds for use in the compositions are those that inhibit falcipain or cruzain with an IC₅₀ of less than about 100 nM, preferably less that 50 nM, more preferably less than 10 nM.

The compounds are preferably formulated into suitable pharmaceutical preparations such as solutions, suspensions, tablets, dispersible tablets, pills, capsules, powders, sustained release formulations or elixirs, for oral administration or in sterile solutions or suspensions for parenteral administration, as well as transdermal patch preparation and dry powder inhalers. Typically the compounds described above are formulated into pharmaceutical compositions using techniques and procedures well known in the art (see, e.g., Ansel Introduction to Pharmaceutical Dosage Forms, Fourth Edition 1985, 126).

In the compositions, effective concentrations of one or more compounds or pharmaceutically acceptable derivatives is (are) mixed with a suitable pharmaceutical carrier or vehicle. The compounds may be derivatized as the corresponding salts, esters, enol ethers or esters, acids, bases, solvates, hydrates or prodrugs prior to formulation, as described above. The concentrations of the compounds in the compositions are effective for delivery of an amount, upon administration, that ameliorates one or more of the symptoms of parasitic infection, particularly malaria or Chagas' disease. Typically, the compositions are formulated for single dosage administration. To formulate a composition, the weight fraction of compound is dissolved, suspended, dispersed or otherwise mixed in a selected vehicle at an effective concentration such that the treated condition is relieved or ameliorated. Pharmaceutical carriers or vehicles suitable for administration of the compounds provided herein include any such carriers known to those skilled in the art to be suitable for the particular mode of administration.

In addition, the compounds may be formulated as the sole pharmaceutically active ingredient in the composition or may be combined with other active ingredients. Liposomal suspensions, including tissue-targeted liposomes, particularly tumor-targeted liposomes, may also be suitable as pharmaceutically acceptable carriers. These may be prepared according to methods known to those skilled in the art. For example, liposome formulations may be prepared as described in U.S. Pat. No. 4,522,811.

The active compound is included in the pharmaceutically acceptable carrier in an amount sufficient to exert a therapeutically useful effect in the absence of undesirable side effects on the patient treated. The therapeutically effective concentration may be determined empirically by testing the compounds in known in vitro and in vivo systems (see, e.g., Rosenthal et al. (1996) Antimicrob. Agents Chemother. 40(7):1600-1603; Dominguez et al. (1997) J. Med. Chem. 40:2726-2732; Clark et al. (1994) Molec. Biochem. Parasitol. 17:129; Ring et al. (1993) Proc. Natl. Acad. Sci. USA 90:3583-3587; Engel et al. (1998) J. Exp. Med. 188(4):725-734; Li et al. (1995) J. Med. Chem. 38:5031) and then extrapolated therefrom for dosages for humans.

The concentration of active compound in the pharmaceutical composition will depend on absorption, inactivation and excretion rates of the active compound, the physicochemical characteristics of the compound, the dosage schedule, and amount administered as well as other factors known to those of skill in the art. For example, the amount that is delivered is sufficient to ameliorate one or more of the symptoms of parasitic infections, particularly malaria or Chagas' disease.

Typically a therapeutically effective dosage should produce a serum concentration of active ingredient of from about 0.1 ng/ml to about 50-100 μg/ml. The pharmaceutical compositions typically should provide a dosage of from about 0.001 mg to about 2000 mg of compound per kilo-gram of body weight per day. Pharmaceutical dosage unit forms are prepared to provide from about 1 mg to about 1000 mg and preferably from about 10 to about 500 mg of the essential active ingredient or a combination of essential ingredients per dosage unit form.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

Preferred pharmaceutically acceptable derivatives include acids, bases, enol ethers and esters, salts, esters, hydrates, solvates and prodrug forms. The derivative is selected such that its pharmacokinetic properties are superior to the corresponding neutral compound.

Thus, effective concentrations or amounts of one or more of the compounds described herein or pharmaceutically acceptable derivatives thereof are mixed with a suitable pharmaceutical carrier or vehicle for systemic, topical or local administration to form pharmaceutical compositions. Compounds are included in an amount effective for ameliorating one or more symptoms of, or for treating or preventing parasitic infections, particularly malaria or Chagas' disease. The concentration of active compound in the composition will depend on absorption, inactivation, excretion rates of the active compound, the dosage schedule, amount administered, particular formulation as well as other factors known to those of skill in the art.

The compositions are intended to be administered by a suitable route, including orally, parenterally, rectally, topically and locally. For oral administration, capsules and tablets are presently preferred. The compositions are in liquid, semi-liquid or solid form and are formulated in a manner suitable for each route of administration. Preferred modes of administration include parenteral and oral modes of administration. Oral administration is presently most preferred.

Solutions or suspensions used for parenteral, intradermal, subcutaneous, or topical application can include any of the following components: a sterile diluent, such as water for injection, saline solution, fixed oil, polyethylene glycol, glycerine, propylene glycol or other synthetic solvent; antimicrobial agents, such as benzyl alcohol and methyl parabens; antioxidants, such as ascorbic acid and sodium bisulfite; chelating agents, such as ethylenediaminetetraacetic acid (EDTA); buffers, such as acetates, citrates and phosphates; and agents for the adjustment of tonicity such as sodium chloride or dextrose. Parenteral preparations can be enclosed in ampules, disposable syringes or single or multiple dose vials made of glass, plastic or other suitable material.

In instances in which the compounds exhibit insufficient solubility, methods for solubilizing compounds may be used. Such methods are known to those of skill in this art, and include, but are not limited to, using cosolvents, such as dimethylsulfoxide (DMSO), using surfactants, such as TWEEN®, or dissolution in aqueous sodium bicarbonate. Derivatives of the compounds, such as prodrugs of the compounds may also be used in formulating effective pharmaceutical compositions.

Upon mixing or addition of the compound(s), the resulting mixture may be a solution, suspension, emulsion or the like. The form of the resulting mixture depends upon a number of factors, including the intended mode of administration and the solubility of the compound in the selected carrier or vehicle. The effective concentration is sufficient for ameliorating the symptoms of the disease, disorder or condition treated and may be empirically determined.

The pharmaceutical compositions are provided for administration to humans and animals in unit dosage forms, such as tablets, capsules, pills, powders, granules, sterile parenteral solutions or suspensions, and oral solutions or suspensions, and oil-water emulsions containing suitable quantities of the compounds or pharmaceutically acceptable derivatives thereof. The pharmaceutically therapeutically active compounds and derivatives thereof are typically formulated and administered in unit-dosage forms or multiple-dosage forms. Unit-dose forms as used herein refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pints or gallons. Hence, multiple dose form is a multiple of unit-doses which are not segregated in packaging.

The composition can contain along with the active ingredient: a diluent such as lactose, sucrose, dicalcium phosphate, or carboxymethylcellulose; a lubricant, such as magnesium stearate, calcium stearate and talc; and a binder such as starch, natural gums, such as gum acaciagelatin, glucose, molasses, polvinylpyrrolidine, celluloses and derivatives thereof, povidone, crospovidones and other such binders known to those of skill in the art. Liquid pharmaceutically administrable compositions can, for example, be prepared by dissolving, dispersing, or otherwise mixing an active compound as defined above and optional pharmaceutical adjuvants in a carrier, such as, for example, water, saline, aqueous dextrose, glycerol, glycols, ethanol, and the like, to thereby form a solution or suspension. If desired, the pharmaceutical composition to be administered may also contain minor amounts of nontoxic auxiliary substances such as wetting agents, emulsifying agents, or solubilizing agents, pH buffering agents and the like, for example, acetate, sodium citrate, cyclodextrine derivatives, sorbitan monolaurate, triethanolamine sodium acetate, triethanolamine oleate, and other such agents. Actual methods of preparing such dosage forms are known, or will be apparent, to those skilled in this art; for example, see Remington's Pharmaceutical Sciences, Mack Publishing Company, Easton, Pa., 15th Edition, 1975. The composition or formulation to be administered will, in any event, contain a quantity of the active compound in an amount sufficient to alleviate the symptoms of the treated subject.

Dosage forms or compositions containing active ingredient in the range of 0.005% to 100% with the balance made up from non-toxic carrier may be prepared. For oral administration, a pharmaceutically acceptable non-toxic composition is formed by the incorporation of any of the normally employed excipients, such as, for example pharmaceutical grades of mannitol, lactose, starch, magnesium stearate, talcum, cellulose derivatives, sodium crosscarmellose, glucose, sucrose, magnesium carbonate or sodium saccharin. Such compositions include solutions, suspensions, tablets, capsules, powders and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparation of these compositions are known to those skilled in the art. The contemplated compositions may contain 0.001%-100% active ingredient, preferably 0.1-85%, typically 75-95%.

The active compounds or pharmaceutically acceptable derivatives may be prepared with carriers that protect the compound against rapid elimination from the body, such as time release formulations or coatings.

The compositions may include other active compounds to obtain desired combinations of properties. The compounds of formula I, or pharmaceutically acceptable derivatives thereof as described herein, may also be advantageously administered for therapeutic or prophylactic purposes together with another pharmacological agent known in the general art to be of value in treating one or more of the diseases or medical conditions referred to hereinabove, such as malaria or Chagas' disease. It is to be understood that such combination therapy constitutes a further aspect of the compositions and methods of treatment provided herein.

1. Compositions for Oral Administration

Oral pharmaceutical dosage forms are either solid, gel or liquid. The solid dosage forms are tablets, capsules, granules, and bulk powders. Types of oral tablets include compressed, chewable lozenges and tablets which may be enteric-coated, sugar-coated or film-coated. Capsules may be hard or soft gelatin capsules, while granules and powders may be provided in non-effervescent or effervescent form with the combination of other ingredients known to those skilled in the art.

In certain embodiments, the formulations are solid dosage forms, preferably capsules or tablets. The tablets, pills, capsules, troches and the like can contain any of the following ingredients, or compounds of a similar nature: a binder; a diluent; a disintegrating agent; a lubricant; a glidant; a sweetening agent; and a flavoring agent.

Examples of binders include microcrystalline cellulose, gum tragacanth, glucose solution, acacia mucilage, gelatin solution, sucrose and starch paste. Lubricants include talc, starch, magnesium or calcium stearate, lycopodium and stearic acid. Diluents include, for example, lactose, sucrose, starch, kaolin, salt, mannitol and dicalcium phosphate. Glidants include, but are not limited to, colloidal silicon dioxide. Disintegrating agents include crosscarmellose sodium, sodium starch glycolate, alginic acid, corn starch, potato starch, bentonite, methylcellulose, agar and carboxymethylcellulose. Coloring agents include, for example, any of the approved certified water soluble FD and C dyes, mixtures thereof; and water insoluble FD and C dyes suspended on alumina hydrate. Sweetening agents include sucrose, lactose, mannitol and artificial sweetening agents such as saccharin, and any number of spray dried flavors. Flavoring agents include natural flavors extracted from plants such as fruits and synthetic blends of compounds which produce a pleasant sensation, such as, but not limited to peppermint and methyl salicylate. Wetting agents include propylene glycol monostearate, sorbitan monooleate, diethylene glycol monolaurate and polyoxyethylene laural ether. Emetic-coatings include fatty acids, fats, waxes, shellac, ammoniated shellac and cellulose acetate phthalates. Film coatings include hydroxyethylcellulose, sodium carboxymethylcellulose, polyethylene glycol 4000 and cellulose acetate phthalate.

If oral administration is desired, the compound could be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it can contain, in addition to material of the above type, a liquid carrier such as a fatty oil. In addition, dosage unit forms can contain various other materials which modify the physical form of the dosage unit, for example, coatings of sugar and other enteric agents. The compounds can also be administered as a component of an elixir, suspension, syrup, wafer, sprinkle, chewing gum or the like. A syrup may contain, in addition to the active compounds, sucrose as a sweetening agent and certain preservatives, dyes and colorings and flavors.

The active materials can also be mixed with other active materials which do not impair the desired action, or with materials that supplement the desired action, such as antacids, H2 blockers, and diuretics. The active ingredient is a compound or pharmaceutically acceptable derivative thereof as described herein. Higher concentrations, up to about 98% by weight of the active ingredient may be included.

Pharmaceutically acceptable carriers included in tablets are binders, lubricants, diluents, disintegrating agents, coloring agents, flavoring agents, and wetting agents. Enteric-coated tablets, because of the enteric-coating, resist the action of stomach acid and dissolve or disintegrate in the neutral or alkaline intestines. Sugar-coated tablets are compressed tablets to which different layers of pharmaceutically acceptable substances are applied. Film-coated tablets are compressed tablets which have been coated with a polymer or other suitable coating. Multiple compressed tablets are compressed tablets made by more than one compression cycle utilizing the pharmaceutically acceptable substances previously mentioned. Coloring agents may also be used in the above dosage forms. Flavoring and sweetening agents are used in compressed tablets, sugar-coated, multiple compressed and chewable tablets. Flavoring and sweetening agents are especially useful in the formation of chewable tablets and lozenges.

Liquid oral dosage forms include aqueous solutions, emulsions, suspensions, solutions and/or suspensions reconstituted from non-effervescent granules and effervescent preparations reconstituted from effervescent granules. Aqueous solutions include, for example, elixirs and syrups. Emulsions are either oil-in-water or water-in-oil.

Elixirs are clear, sweetened, hydroalcoholic preparations. Pharmaceutically acceptable carriers used in elixirs include solvents. Syrups are concentrated aqueous solutions of a sugar, for example, sucrose, and may contain a preservative. An emulsion is a two-phase system in which one liquid is dispersed in the form of small globules throughout another liquid. Pharmaceutically acceptable carriers used in emulsions are non-aqueous liquids, emulsifying agents and preservatives. Suspensions use pharmaceutically acceptable suspending agents and preservatives. Pharmaceutically acceptable substances used in non-effervescent granules, to be reconstituted into a liquid oral dosage form, include diluents, sweeteners and wetting agents. Pharmaceutically acceptable substances used in effervescent granules, to be reconstituted into a liquid oral dosage form, include organic acids and a source of carbon dioxide. Coloring and flavoring agents are used in all of the above dosage forms.

Solvents include glycerin, sorbitol, ethyl alcohol and syrup. Examples of preservatives include glycerin, methyl and propylparaben, benzoic add, sodium benzoate and alcohol. Examples of non-aqueous liquids utilized in emulsions include mineral oil and cottonseed oil. Examples of emulsifying agents include gelatin, acacia, tragacanth, bentonite, and surfactants such as polyoxyethylene sorbitan monooleate. Suspending agents include sodium carboxymethylcellulose, pectin, tragacanth, Veegum and acacia. Diluents include lactose and sucrose. Sweetening agents include sucrose, syrups, glycerin and artificial sweetening agents such as saccharin. Wetting agents include propylene glycol monostearate, sorbitan monooleate, diethylene glycol monolaurate and polyoxyethylene lauryl ether. Organic adds include citric and tartaric acid. Sources of carbon dioxide include sodium bicarbonate and sodium carbonate. Coloring agents include any of the approved certified water soluble FD and C dyes, and mixtures thereof. Flavoring agents include natural flavors extracted from plants such fruits, and synthetic blends of compounds which produce a pleasant taste sensation.

For a solid dosage form, the solution or suspension, in for example propylene carbonate, vegetable oils or triglycerides, is preferably encapsulated in a gelatin capsule. Such solutions, and the preparation and encapsulation thereof, are disclosed in U.S. Pat. Nos 4,328,245; 4,409,239; and 4,410,545. For a liquid dosage form, the solution, e.g., for example, in a polyethylene glycol, may be diluted with a sufficient quantity of a pharmaceutically acceptable liquid carrier, e.g., water, to be easily measured for administration.

Alternatively, liquid or semi-solid oral formulations may be prepared by dissolving or dispersing the active compound or salt in vegetable oils, glycols, triglycerides, propylene glycol esters (e.g., propylene carbonate) and other such carriers, and encapsulating these solutions or suspensions in hard or soft gelatin capsule shells. Other useful formulations include those set forth in U.S. Pat. Nos. Re 28,819 and 4,358,603.

In all embodiments, tablets and capsules formulations may be coated as known by those of skill in the art in order to modify or sustain dissolution of the active ingredient. Thus, for example, they may be coated with a conventional enterically digestible coating, such as phenylsalicylate, waxes and cellulose acetate phthalate.

2. Injectables, Solutions and Emulsions

Parenteral administration, generally characterized by injection, either subcutaneously, intramuscularly or intravenously is also contemplated herein. Injectables can be prepared in conventional forms, either as liquid solutions or suspensions, solid forms suitable for solution or suspension in liquid prior to injection, or as emulsions. Suitable excipients are, for example, water, saline, dextrose, glycerol or ethanol. In addition, if desired, the pharmaceutical compositions to be administered may also contain minor amounts of non-toxic auxiliary substances such as wetting or emulsifying agents, pH buffering agents, stabilizers, solubility enhancers, and other such agents, such as for example, sodium acetate, sorbitan monolaurate, triethanolamine oleate and cyclodextrins. Implantation of a slow-release or sustained-release system, such that a constant level of dosage is maintained (see, e.g., U.S. Pat. No. 3,710,795) is also contemplated herein. The percentage of active compound contained in such parenteral compositions is highly dependent on the specific nature thereof, as well as the activity of the compound and the needs of the subject.

Parenteral administration of the compositions includes intravenous, subcutaneous and intramuscular administrations. Preparations for parenteral administration include sterile solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use and sterile emulsions. The solutions may be either aqueous or nonaqueous.

If administered intravenously, suitable carriers include physiological saline or phosphate buffered saline (PBS), and solutions containing thickening and solubilizing agents, such as glucose, polyethylene glycol, and polypropylene glycol and mixtures thereof.

Pharmaceutically acceptable carriers used in parenteral preparations include aqueous vehicles, nonaqueous vehicles, antimicrobial agents, isotonic agents, buffers, antioxidants, local anesthetics, suspending and dispersing agents, emulsifying agents, sequestering or chelating agents and other pharmaceutically acceptable substances.

Examples of aqueous vehicles include Sodium Chloride Injection, Ringers Injection, Isotonic Dextrose Injection, Sterile Water Injection, Dextrose and Lactated Ringers Injection. Nonaqueous parenteral vehicles include fixed oils of vegetable origin, cottonseed oil, corn oil, sesame oil and peanut oil. Antimicrobial agents in bacteriostatic or fungistatic concentrations must be added to parenteral preparations packaged in multiple-dose containers which include phenols or cresols, mercurials, benzyl alcohol, chlorobutanol, methyl and propyl p-hydroxybenzoic acid esters, thimerosal, benzalkonium chloride and benzethonium chloride. Isotonic agents include sodium chloride and dextrose. Buffers include phosphate and citrate. Antioxidants include sodium bisulfate. Local anesthetics include procaine hydrochloride. Suspending and dispersing agents include sodium carboxymethylcelluose, hydroxypropyl methylcellulose and polyvinylpyrrolidone. Emulsifying agents include Polysorbate 80 (TWEEN® 80). A sequestering or chelating agent of metal ions include EDTA. Pharmaceutical carriers also include ethyl alcohol, polyethylene glycol and propylene glycol for water miscible vehicles and sodium hydroxide, hydrochloric acid, citric acid or lactic acid for pH adjustment.

The concentration of the pharmaceutically active compound is adjusted so that an injection provides an effective amount to produce the desired pharmacological effect. The exact dose depends on the age, weight and condition of the patient or animal as is known in the art.

The unit-dose parenteral preparations are packaged in an ampoule, a vial or a syringe with a needle. All preparations for parenteral administration must be sterile, as is known and practiced in the art.

Illustratively, intravenous or intraarterial infusion of a sterile aqueous solution containing an active compound is an effective mode of administration. Another embodiment is a sterile aqueous or oily solution or suspension containing an active material injected as necessary to produce the desired pharmacological effect.

Injectables are designed for local and systemic administration. Typically a therapeutically effective dosage is formulated to contain a concentration of at least about 0.1% w/w up to about 90% w/w or more, preferably more than 1% w/w of the active compound to the treated tissue(s). The active ingredient may be administered at once, or may be divided into a number of smaller doses to be administered at intervals of time. It is understood that the precise dosage and duration of treatment is a function of the tissue being treated and may be determined empirically using known testing protocols or by extrapolation from in vivo or in vitro test data. It is to be noted that concentrations and dosage values may also vary with the age of the individual treated. It is to be further understood that for any particular subject, specific dosage regimens should be adjusted over time according to the individual need and the professional judgment of the person administering or supervising the administration of the formulations, and that the concentration ranges set forth herein are exemplary only and are not intended to limit the scope or practice of the claimed formulations.

The compound may be suspended in micronized or other suitable form or may be derivatized to produce a more soluble active product or to produce a prodrug. The form of the resulting mixture depends upon a number of factors, including the intended mode of administration and the solubility of the compound in the selected carrier or vehicle. The effective concentration is sufficient for ameliorating the symptoms of the condition and may be empirically determined.

3. Lyophilized Powders

Of interest herein are also lyophilized powders, which can be reconstituted for administration as solutions, emulsions and other mixtures. They may also be reconstituted and formulated as solids or gels.

The sterile, lyophilized powder is prepared by dissolving a compound of formula I in a suitable solvent. The solvent may contain an excipient which improves the stability or other pharmacological component of the powder or reconstituted solution, prepared from the powder. Excipients that may be used include, but are not limited to, dextrose, sorbital, fructose, corn syrup, xylitol, glycerin, glucose, sucrose or other suitable agent. The solvent may also contain a buffer, such as citrate, sodium or potassium phosphate or other such buffer known to those of skill in the art at, typically, about neutral pH. Subsequent sterile filtration of the solution followed by lyophilization under standard conditions known to those of skill in the art provides the desired formulation. Generally, the resulting solution will be apportioned into vials for lyophilization. Each vial will contain a single dosage (10-1000 mg, preferably 100-500 mg) or multiple dosages of the compound. The lyophilized powder can be stored under appropriate conditions, such as at about 4° C. to room temperature.

Reconstitution of this lyophilized powder with water for injection provides a formulation for use in parenteral administration. For reconstitution, about 1-50 mg, preferably 5-35 mg, more preferably about 9-30 mg of lyophilized powder, is added per mL of sterile water or other suitable carrier. The precise amount depends upon the selected compound. Such amount can be empirically determined.

4. Topical Administration

Topical mixtures are prepared as described for the local and systemic administration. The resulting mixture may be a solution, suspension, emulsions or the like and are formulated as creams, gels, ointments, emulsions, solutions, elixirs, lotions, suspensions, tinctures, pastes, foams, aerosols, irrigations, sprays, suppositories, bandages, dermal patches or any other formulations suitable for topical administration.

The compounds or pharmaceutically acceptable derivatives thereof may be formulated as aerosols for topical application, such as by inhalation (see, e.g., U.S. Pat. Nos. 4,044,126, 4,414,209, and 4,364,923, which describe aerosols for delivery of a steroid useful for treatment inflammatory diseases, particularly asthma). These formulations for administration to the respiratory tract can be in the form of an aerosol or solution for a nebulizer, or as a microfine powder for insufflation, alone or in combination with an inert carrier such as lactose. In such a case, the particles of the formulation will typically have diameters of less than 50 microns, preferably less than 10 microns.

The compounds may be formulated for local or topical application, such as for topical application to the skin and mucous membranes, such as in the eye, in the form of gels, creams, and lotions and for application to the eye or for intracisternal or intraspinal application. Topical administration is contemplated for transdermal delivery and also for administration to the eyes or mucosa, or for inhalation therapies. Nasal solutions of the active compound alone or in combination with other pharmaceutically acceptable excipients can also be administered.

These solutions, particularly those intended for ophthalmic use, may be formulated as 0.01%-10% isotonic solutions, pH about 5-7, with appropriate salts.

5. Compositions for Other Routes of Administration

Other routes of administration, such as transdermal patches and rectal administration are also contemplated herein.

For example, pharmaceutical dosage forms for rectal administration are rectal suppositories, capsules and tablets for systemic effect. Rectal suppositories are used herein mean solid bodies for insertion into the rectum which melt or soften at body temperature releasing one or more pharmacologically or therapeutically active ingredients. Pharmaceutically acceptable substances utilized in rectal suppositories are bases or vehicles and agents to raise the melting point. Examples of bases include cocoa butter (theobroma oil), glycerin-gelatin, carbowax (polyoxyethylene glycol) and appropriate mixtures of mono-, di- and triglycerides of fatty acids. Combinations of the various bases may be used. Agents to raise the melting point of suppositories include spermaceti and wax. Rectal suppositories may be prepared either by the compressed method or by molding. The typical weight of a rectal suppository is about 2 to 3 gm.

Tablets and capsules for rectal administration are manufactured using the same pharmaceutically acceptable substance and by the same methods as for formulations for oral administration.

6. Combination Therapy

Also contemplated herein are compositions for use in the methods containing (i) a compound provided herein, or a pharmaceutically acceptable derivative thereof, and (ii) a known antiparasitic compound or composition. The antiparasitic compound or composition may be any known to those of skill in the art, including marketed and experimental therapeutics. Many such compounds are well known to those of skill in the art (see, e.g., Rosenthal (1998) Emerging Infectious Diseases 4(1):49-57; Rosenthal et al. (1996) Antimicrob. Agents Chemother. 40(7):1600-1603; Dominguez et al. (1997) J. Med. Chem. 40:2726-2732; Li et al. (1996) Bioorg. Med. Chem. 4(9):1421-1427; Ring et al. (1993) Proc. Natl. Acad. Sci. USA 90:3583-3587; and International Patent Application Publication Nos. WO 97/30072, WO 96/40647 and WO 96/40737; see also, Engel et al. (1998) J. Exp. Med. 188(4):725-734). The compositions described above may be more efficacious due to a synergistic effect between the compound provided herein and the known antiparasitic compound or composition. In such cases, the compositions described above may be particularly useful in the treatment of resistant strains of parasitic infection.

Among the known antiparasitic agents for use in this embodiment are chloroquine, quinine, quinidine, amodiaquine, mefloquine, sulfadoxine, pyrimethamine, a tetracyline antibiotic, clindamycin, a sulfa antibiotic, doxycyline, proguanil, dapsone, primaquine, artemisinin, artesunate, artelinate, artemether, arteether, dihydroartemisinin, halofantrine, atovaquione, pyronaridine, desferrioxamine, azithromycin, SC-50083, Ro 40-4388, “compound 7”, ((benzyloxycarbonyl)phenylalanyl)arginyl fluoromethyl ketone, ((morpholinocarbonyl)phenylalanyl)homophenylalanyl fluoromethyl ketone, (((morpholinocarbonyl)leucyl)homophenylalanyl)vinyl phenyl sulfone, oxalic bis((2-hydroxy-1-naphthylmethylene)hydrazide), 1-(2,5-dichlorophenyl)-3-(4-quinolinyl)-2-propen-1-one, and 7-chloro-1,2-dihydro-2-(2,3-dimethoxyphenyl)-5,5-dioxide-4-(1H, 10H)-phenothiazinone.

Other known antiparasitic agents for use in this embodiment include nifurtimox, benznidazole, (((morpholinocarbonyl)phenylalanyl)-homophenylalanyl)vinyl phenyl sulfone, (((morpholinocarbonyl)phenyl-alanyl)lysyl)vinyl phenyl sulfone, (((morpholinocarbonyl)phenylalanyl)-valyl)vinyl phenyl sulfone, (((morpholinocarbonyl)phenylalanyl)-O-benzylseryl)vinyl phenyl sulfone, (((morpholinocarbonyl)leucyl)-homophenylalanyl)vinyl phenyl sulfone, (((morpholinocarbonyl)tyrosyl)-homophenylalanyl)vinyl phenyl sulfone, (((tert-butoxycarbonyl)-2-tetrahydroisoquinolylcarbonyl)homophenylalanyl) phenyl vinyl sulfone, (((morpholinocarbonyl)tyrosyl)homophenylalanyl)vinylphenyl sulfone, (((morpholinocarbonyl)phenylalanyl)homophenylalanyl fluromethylketone and (((morpholinocarbonyl)phenylalanyl)homophenylalanyl)valine benzylamide.

7. Articles of Manufacture

The compounds or pharmaceutically acceptable derivatives may be packaged as articles of manufacture containing packaging material, a compound or pharmaceutically acceptable derivative thereof provided herein, which is effective for inhibiting falcipain or cruzain, or for treatment, prevention or amelioration of one or more symptoms of parasitic infections, particularly malaria or Chagas' disease, and a label that indicates that the compound or pharmaceutically acceptable derivative thereof is used for inhibiting falcipain or cruzain, or for treatment, prevention or amelioration of one or more symptoms of parasitic infections, particularly malaria or Chagas' disease.

The articles of manufacture provided herein contain packaging materials. Packaging materials for use in packaging pharmaceutical products are well known to those of skill in the art. See, e.g., U.S. Pat. Nos. 5,323,907, 5,052,558 and 5,033,352. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety treatments for any disorder in which falcipain is implicated as a mediator or contributor to the symptoms or cause.

E. Evaluation of the Activity of the Compounds

Standard physiological, pharmacological and biochemical procedures are available for testing the compounds to identify those that possess biological activities that interfere with, antagonize, inhibit, or otherwise modulate the activity of falcipain or cruzain. For example, the properties of a potential inhibitor may be assessed as a function of its ability to inhibit falcipain or cruzain including the ability in vitro to antagonize the activity of falcipain or cruzain.

Assays that may be used to evaluate falcipain activity are well known to those of skill in the art. See, e.g., Rosenthal et al. (1996) Antimicrob. Agents Chemother. 40(7):1600-1603; Dominguez et al. (1997) J. Med. Chem. 40:2726-2732; Clark et al. (1994) Molec. Biochem. Parasitol. 17:129; Ring et al. (1993) Proc. Natl. Acad. Sci. USA 90:3583-3587.

Assays that may be used to evaluate cruzain activity and effectiveness in treatment of Chagas' disease are also well known to those of skill in the art. See, e.g., Eakin et al. (1992) J. Biol. Chem. 267(11): 7411-7420; Engel et al. (1 998) J. Exp. Med. 188(4):725-734; Li et al. (1995) J. Med. Chem. 38:5031.

Briefly, In vitro assays compare the rate of hydrolysis of the substrate Z-Phe-Arg-AMC by either falcipain or cruzain after pretreatment by a compound provided herein with untreated enzyme as the control. These fluorometric assays are routinely performed in a 96-well format, providing adequate throughput for the studies.

An in vitro method of assaying for effectiveness in treatment of Chagas' disease involves culturing irradiated (3000 rad) J774 macrophages in RPMI-1640 medium with 5% heat-inactivated FCS (RPMI medium) for 24 h at 37° C. After infection with T cruzi trypomastigotes of the Y strain for 3 h, monolayers are washed with RPMI medium and stocks are made at 20 mM in DMSO and all assays include DMSO (0.01-0.02%, vol.vol) controls. The test compound is evaluated in T cruzi-infected macrophage cultures for 21-30 d. Trypomastigote output, indicative of the completion of the intracellular cycle, is then assayed in treated and untreated cultures to determin growth inhibition of intracellular T. cruzi amastigotes. After this initial inhibitor screen, T. cruzi-infected macrophages are treated with the test compound for up to 76 h. Monolayers are washed, fixed with 4% paraformaldehyde, and then Giemsa stained at determined intervals. To evaluate treatment, the percentage of infected macrophages and the total number of intracellular amastigotes in 100 infected macrophages is quantified. A decrease in the number of intracellular generation indicates inhibition of intracellular growth of T. cruzi amastigotes and is calculated from the total number of intracellular amastigotes per 100 infected macrophages.

Using such assays, the relative abilities of the compounds provided herein to inhibit or otherwise modulate the activity of falcipain or cruzain have been and can be assessed. Those that possess the desired in vitro properties, such as specific inhibition of falcipain or cruzain, are selected. The selected compounds that exhibit desirable activities may be therapeutically useful in the methods described herein and are tested for such uses employing the above-described assays from which the in vivo effectiveness may be evaluated. Compounds that exhibit the in vitro activities that correlate with the in vivo effectiveness will then be formulated in suitable pharmaceutical compositions and used as therapeutics.

F. Methods of use of Falcipain and Cruzain Inhibitors

Falcipain has been implicated in the growth and development of P. falciparum, and thus in the development and progression of malaria. Cruzain has been implicated in the development of T. cruzi, and thus in the development and progression of Chagas' disease. Methods using therapeutically effective concentrations one or more of the compounds of formula I, or pharmaceutically acceptable derivatives thereof, for treating, preventing or ameliorating one or more symptoms of parasitic infections, particularly malaria or Chagas' disease, are provided herein.

Preferably, a medicament containing the compound is administered orally, although administration by other methods, such as, but not limited to, topical, perenteral, intravenous (IV) and local administration may be tolerated in some instances. In certain cases, the medicament containing the compound is injected into the circulatory system of a subject in order to deliver a dose to the targeted cells. Targeting may be effected by linking the compound to a targeting agent specific for the desired cells, such as, but not limited to, cells associated with the malaria parasite. See, e.g., U.S. Pat. Nos. 5,456,663, 4,764,359, 5,543,391, 5,820,879, 5,026,558. Dosages may be determined empirically, but will typically be in the range of about 0.01 mg to about 100 mg of the compound per kilogram of body weight as a daily dosage.

Methods of inhibiting the development or growth of parasites, particularly malarial parasites or parasites that are the causative agent of Chagas' disease, more particularly Plasmodium falciparum, Trypanosoma cruzi or Trypanosoma brucei, are also provided.

The following examples are included for illustrative purposes only and are not intended to limit the scope of the invention.

### EXAMPLE 1

### Preparation of Methyl (E)-4-((N-(benzyloxycarbonyl)phenylalaninyl)amino)-6-phenyl-2-hexenoate

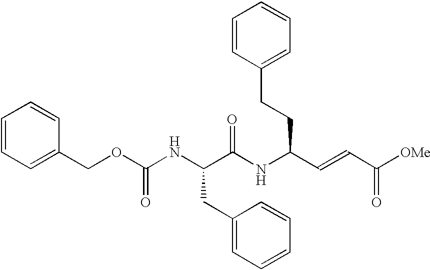

### Step A: N-Methoxy-N-methyl-2-( N-(tert-butoxycarbonyl )amino)-4-phenylbutanamide

Isobutyl chloroformate (2.05 mL, 0.016 mol) was added to a solution of (S)-2-(N-(tert-butoxycarbonyl)amino)-4-phenylbutanoic acid (4.4 g, 0.016 mol) and 4-methyl morpholine (3.5 mL, 0.032 mol) in DCM (160 mL) at 0° C. The reaction mixture was stirred at 0° C. for 15 min. and then N,O-dimethylhydroxylamine hydrochloride (1.54 g, 0.016 mol) was added. The resulting solution was stirred at 0° C. for 20 min and at 25° C. for 30 min and then was partitioned between water (100 mL) and EtOAc (2×100 mL). The combined organic layers were dried over sodium sulfate and were concentrated. Purification of the residue by flash chromatography using EtOAc/hexane as eluent provided the title compound (4.52 g, 89%) as colorless oil. R_{f} 0.7 (EtOAc/hexane 1/1); ¹H NMR (CDCl₃) δ1.45 (s, 9H), 1.78-1.90 (m, 1H), 1.98-2.08 (m, 1H), 2.60-2.80 (m, 2H), 3.17 (s, 3H), 3.62 (s, 3H), 4.62-4.72 (m, 1H), 5.22 (br s, 1H), 7.12-7.32 (m, 5H).

### Step B: Methyl (E)-4-((N-tert-butoxycarbonyl)amino)-6-phenyl-2-hexenoate

Lithium aluminum hydride (31.5 mL of a 1.0 M solution in THF, 0.032 mol) was added to a solution of N-methoxy-N-methyl-2-(N-(tert-butoxycarbonyl)amino)-4-phenylbutanamide (4.52 g, 0.014 mol) in THF (100 mL) at −78° C., and the reaction mixture was stirred at −78° C. for 1 h. MeOH (10 mL) and a saturated solution of Rochelle salt were added sequentially, and the mixture was warmed to 25° C. The resulting suspension was diluted with ether (200 mL) and was washed with brine. The organic layer was dried over sodium sulfate and concentrated under reduced pressure.

To a solution of the crude aldehyde in DCM (100 mL) was added methyl (triphenylphosphoranylidene)acetate (7.02 g, 0.021 mol). The resulting solution was stirred at RT for 48 h, then was concentrated and the crude residue purified by flash chromatography on silica gel (eluting with EtOAc/hexane) to afford the title compound (3.78 g, 84%) as a white solid. R_{f} 0.6 (EtOAc/hexane 3/7); ¹H NMR (CDCl₃) δ1.45 (s, 9H), 1.78-1.95 (m, 2H), 2.62-2.76 (m, 2H), 3.74 (s, 3H), 4.30-4.40 (m, 1H), 4.50-4.60 (m, 1H), 5.93 (d, J=15.6 Hz, 1H), 6.87 (dd, J=5.2 and 15.6 Hz, 1H), 7.14-7.31 (m, 5H).

### Step C: Methyl (E)-4-((N-(benzyloxycarbonyl)phenylalaninyl)amino)-6-phenyl-2-hexenoate

Methyl (E)-4-((N-tert-butoxycarbonyl)amino)-6-phenyl-2-hexenoate (0.5 g, 1.57 mmol) was added to a solution of concentrated HCl (3 mL) in MeOH (6 mL). The resulting solution was stirred at 25° C. for 3 h then was concentrated under reduced pressure.

To a solution of the obtained crude amine in DMF (20 mL) were added EDC (0.36 g, 1.88 mmol), HOBt (0.25 g, 1.88 mmol), N-benzyloxycarbonylphenylalanine (0.56 g, 1.88 mmol) and 4-methyl morpholine (0.86 mL, 7.82 mmol). The resulting solution was stirred at 25° C. for 18 h then was partitioned between water (100 mL) and EtOAc (2×100 mL). The combined organic layers were washed with HCl 1.0 M (100 mL), water (100 mL), saturated solution of NaHCO₃, and brine (100 mL). The organic solution was dried over sodium sulfate and concentrated. Purification of the residue by flash chromatography using EtOAc/hexane as eluent provided the title compound (0.51 g, 65%) as a white solid. R_{f} 0.3 (EtOAc/hexane 3/7); ¹H NMR (CDCl₃) δ1.70-1.88 (m, 2H), 2.53 (t, J=8.0 Hz, 2H), 2.96-3.10 (m, 2H), 3.73 (s, 3H), 4.30-4.40 (m, 1H), 4.50-4.60 (m, 1H), 5.06 (s, 2H), 5.31-5.40 (m, 1H), 5.67 (d, J=15.6 Hz, 1H), 5.90-6.00 (m, 1H), 6.66 (dd, J=6.0 and 16.0 Hz, 1H), 7.02-7.40 (m, 15 H).

### EXAMPLE 2

### Preparation of Methyl (E)-4-((N-(benzyloxycarbonyl)phenylalaninyl)amino)-5-phenyl-2-pentenoate

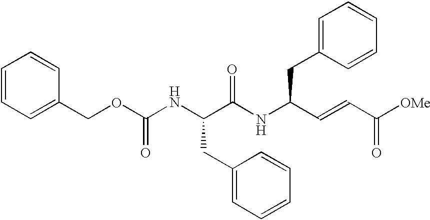

### Step A: Methyl (E)-4-((N-tert-butoxycarbonyl)amino)-5-phenyl-2-pentenoate

Diisobutylaluminum hydride (34 mL of a 1.0 M solution in hexane, 0.033 mol) was added dropwise to a solution of N-tert-butoxycarbonyl-phenylalanine methyl ester (7.1 g, 0.025 mol) in toluene (100 mL) at −78° C., and the reaction mixture was stirred at −78° C. for 1 h. MeOH (5 mL) and a saturated solution of Rochelle salt were added sequentially, and the mixture was warmed to 25° C. The resulting suspension extracted with ether (3×100 mL). The combined organic layers were dried over sodium sulfate and were concentrated. Purification of the residue by flash chromatography using EtOAc/hexane as eluent provided the aldehyde intermediate (4.5 g, 71%) as colorless oil. R_{f} 0.7 (EtOAc/hexane 3/7).

To a solution of the aldehyde (4.0 g, 0.014 mol) in DCM (100 mL) was added methyl (triphenylphosphoranylidene)acetate (6.0 g, 0.018 mol). The resulting solution was stirred at 25° C. for 2 h, then was concentrated and the crude residue purified by flash chromatography on silica gel (eluting with EtOAc/hexane) to afford the title compound (3.78 g, 84%) as a white solid. R_{f} 0.75 (EtOAc/hexane 3/7); ¹H NMR (CDCl₃) δ1.40 (s, 9H), 2.85-2.95 (m, 2H), 3.72 (s, 3H), 4.50-4.70 (m, 2H), 5.85 (d, J=15.6 Hz, 1H), 6.91 (dd, J=5.2 and 15.6 Hz, 1H), 7.10-7.35 (m, 5H).

### Step B: Methyl (E)-4-((N-(benzyloxycarbonyl)phenylalaninyl)amino)-5-phenyl-2-pentenoate

Methyl (E)-4-((N-tert-butoxycarbonyl)amino)-5-phenyl-2-pentenoate (2.5 g, 8.19 mmol) was added to a solution of HCl conc. (5 mL) in MeOH (15 mL). The resulting solution was stirred at 25° C. for 3 h then was concentrated under reduced pressure to afford the crude amine intermediate as white solid.

To a solution of the above amine in DMF (20 mL) were added EDC (1.90 g, 9.8 mmol), HOBt (1.33 g, 9.8 mmol), Z-Phe (2.94 g, 9.8 mmol) and 4-methyl morpholine (4.5 mL, 40.9 mmol). The resulting solution was stirred at 25° C. for 18 h then was partitioned between water (100 mL) and EtOAc (2×100 mL). The combined organic layers were washed with HCl 1.0 M (100 mL), water (100 mL), saturated solution of NaHCO₃, and brine (100 mL). The organic solution was dried over sodium sulfate and concentrated. Purification of the residue by flash chromatography using EtOAc/hexane as eluent provided the title compound (2.0 g, 50%) as a white solid. R_{f} 0.7 (EtOAc/hexane 1/1); ¹H NMR (CDCl₃) δ2.70-2.85 (m, 2H), 2.96-3.10 (m, 2H), 3.72 (s, 3H), 4.30-4.40 (m, 1H), 4.80-4.90 (m, 1H), 5.07 (s, 2H), 5.10-5.25 (m, 1H), 5.57 (d, J=15.6 Hz, 1H), 5.72-5.81 (m, 1H), 6.72 (dd, J=6.0 and 16.0 Hz, 1H), 7.02-7.40 (m, 15 H).

### EXAMPLE 3

### Preparation of N-(1-indolinyl)-(E)-4-((N-(tert-butoxycarbonyl)phenyl-alaninyl)amino)-6-phenyl-2-hexenamide

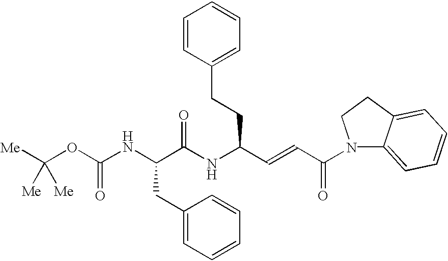

### Step A: (E)-4-((N-tert-Butoxycarbonyl)amino)-6-phenyl-2-hexenoic acid

To a solution of methyl (E)-4-((N-tert-butoxycarbonyl)amino)-6-phenyl-2-hexenoate (1.0 g, 3.13 mmol) in ETOH (20 mL) was added NAOH (7.2 mL of a 1.0 M solution in water, 7.20 mmol). The resulting solution was stirred at 25° C. for 3 h, then was concentrated and diluted with water (50 mL). The aqueous solution was washed with ether (2×50 mL), acidified with HCl conc. and extracted with chloroform (2×50 mL). The collected organic phase was dried and concentrated under reduced pressure to afford the title compound (0.95 g, 98%) as a white foam. ¹H NMR (CDCl₃) δ1.45 (s, 9H), 1.78-1.95 (m, 2H), 2.62-2.80 (m, 2H), 4.30-4.40 (m, 1H), 4.50-4.60 (m, 1H), 5.93 (d, J=15.6 Hz, 1H), 6.90-7.00 (m, 1H), 7.14-7.35 (m, 5H).

### Step B: N-(1-indolinyl)-(E)-4-((N-tert-Butoxycarbonyl)amino)-6-phenyl-2-hexenamide

To a solution of (E)-4-((N-tert-butoxycarbonyl)amino)-6-phenyl-2-hexenoic acid (0.95 g, 3.13 mmol) in DMF (20 mL) were added indoline (0.42 mL, 3.76 mmol), EDC (0.72 g, 3.76 mmol), HOAt (0.51 g, 3.76 mmol), and 4-methyl morpholine (1.02 mL, 9.39 mmol). The resulting solution was stirred at 25° C. for 18 h then was partitioned between water (100 mL) and EtOAc (2×100 mL). The combined organic layers were washed with HCl 1.0 M (100 mL), water (100 mL), saturated solution of NaHCO₃, and brine (100 mL). The organic solution was dried over sodium sulfate and concentrated. Purification of the residue by flash chromatography using EtOAc/hexane as eluent provided the title compound (1.1 g, 86%) as a white solid. R_{f} 0.6 (EtOAc/hexane 1/4); ¹H NMR (CDCl₃) δ1.46 (s, 9H), 1.80-2.00 (m, 2H), 2.62-2.80 (m, 2H), 3.15-3.25 (m, 2H), 4.10-4.20 (m, 2H), 4.30-4.40 (m, 1H), 4.55-4.65 (m, 1H), 6.34 (d, J=15.0 Hz, 1H), 6.83-6.95 (m, 1H), 7.02 (t, J=7.2 Hz, 1H), 7.12-7.35 (m, 7H), 8.27 (d, J=7.2 Hz, 1H).

### Step C: N-(1-indolinyl)-(E)-4-((N-(tert-butoxycarbonyl)phenylalaninyl)-amino)-6-phenyl-2-hexenamide

N-(1-indonlinyl)-(E)-4-((N-tert-Butoxycarbonyl)amino)-6-phenyl-2-hexenamide (0.42 g, 1.04 mmol) was added to a 4M solution of HCl in dioxane (10 mL). The resulting solution was stirred at 25° C. for 3 h then was concentrated under reduced pressure to afford the amine intermediate as a white solid.

To a solution of the above intermediate in DMF (10 mL) were added EDC (0.24 g, 1.23 mmol), HOAt (0.17 g, 1.23 mmol), N-(tert-butoxycarbonyl)phenylalanine (0.41 g, 1.54 mmol) and 4-methyl morpholine (0.57 mL, 5.16 mmol). The resulting solution was stirred at 25° C. for 18 h then was partitioned between water (50 mL) and EtOAc (2×50 mL). The combined organic layers were washed with HCl 1.0 M (50 mL), water (50 mL), saturated solution of NaHCO₃ (50 mL), and brine (500 mL). The organic solution was dried over sodium sulfate and concentrated. Purification of the residue by flash chromatography using EtOAc/hexane as eluent provided the title compound (0.31 g, 54%) as white foam. R_{f} 0.3 (EtOAc/hexane 3/7); ¹H NMR (CDCl₃) δ1.40 (s, 9H), 1.80-2.00 (m, 2H), 2.63 (t, J=7.6 Hz, 2H), 3.01-3.15 (m, 2H), 3.15-3.23 (m, 2H), 4.01-4.18 (m, 2H), 4.30-4.40 (m, 1H), 4.60-4.70 (m, 1H), 4.75-4.85 (m, 1H), 5.90-6.00 (m, 1H), 6.24 (d, J=15.4 Hz, 1H), 6.75-6.85 (m, 1H), 7.03 (t, J=7.2 Hz, 1H), 7.10-7.35 (m, 12H), 8.22 (brs, 1H); MS (m/z): 554 (M+1).

### EXAMPLE 4

### Preparation of N-(1-indolinyl)-(E)-4-((N-tert-butoxycarbonyl-S,S-dioxomethioninyl)amino)-6-phenyl-2-hexenoate

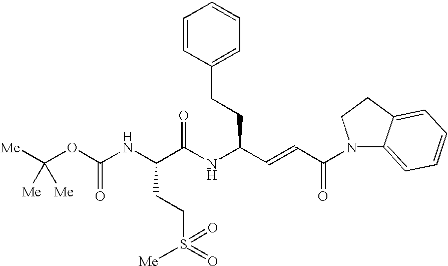

N-(1-Indolinyl)-(E)-4-((N-tert-butoxycarbonyl)amino)-6-phenyl-2-hexenamide (0.42 g, 1.04 mmol) was added to a 4M solution of HCl in dioxane (10 mL). The resulting solution was stirred at 25° C. for 3 h then was concentrated under reduced pressure to afford the amine intermediate as a white solid.

To a solution of the above intermediate in DMF (10 mL) were added EDC (0.24 g, 1.23 mmol), HOAt (0.17 g, 1.23 mmol), N-(tert-butoxycarbonyl-S,S-dioxomethionine (0.44 g, 1.54 mmol) and 4-methyl morpholine (0.57 mL, 5.16 mmol). The resulting solution was stirred at 25° C. for 18 h then was partitioned between water (50 mL) and EtOAc (2×50 mL). The combined organic layers were washed with HCl 1.0 M (50 mL), water (50 mL), saturated solution of NaHCO₃ (50 mL), and brine (500 mL). The organic solution was dried over sodium sulfate and concentrated. Purification of the residue by flash chromatography using EtOAc/hexane as eluent provided the title compound (0.30 g, 51%) as white foam. R_{f} 0.3 (EtOAc/hexane 7/3); ¹H NMR (CDCl₃) δ1.45 (s, 9H), 1.90-2.06 (m, 2H), 2.12-2.26 (m, 2H), 2.28-3.00 (m, 2H), 2.73 (t, J=7.6 Hz, 2H), 2.95-3.30 (m, 7H), 4.10-4.20 (m, 2H), 4.30-4.40 (m, 1H), 4.65-4.75 (m, 1H), 5.20-5.30 (m, 1H), 6.40 (d, J=15.4 Hz, 1H), 6.55-6.63 (m, 1H), 6.90-7.00 (m, 1H), 7.06 (t, J=7.2 Hz, 1H), 7.10-7.40 (m, 8H), 8.22 (brs, 1H); MS (m/z): 570 (M+1).

### EXAMPLE 5

### Preparation of N-(1-indolinyl)-(E)-4-((N-(tert-butoxycarbonyl)leucyl)amino)-6-phenyl-2-hexenamide

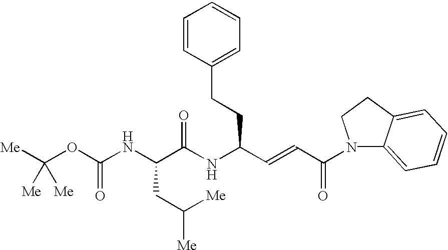

N-(1-Indolinyl)-(E)-4-((N-tert-butoxycarbonyl)amino)-6-phenyl-2-hexenamide (0.42 g, 1.04 mmol) was added to a 4M solution of HCl in dioxane (10 mL). The resulting solution was stirred at 25° C. for 3 h then was concentrated under reduced pressure to afford the amine intermediate as a white solid.

To a solution of the above intermediate in DMF (10 mL) were added EDC (0.24 g, 1.23 mmol), HOAt (0.17 g, 1.23 mmol), N-(tert-butoxycarbonyl)leucine (0.27 g, 1.08 mmol) and 4-methyl morpholine (0.57 mL, 5.16 mmol). The resulting solution was stirred at 25° C. for 18 h then was partitioned between water (50 mL) and EtOAc (2×50 mL). The combined organic layers were washed with HCl 1.0 M (50 mL), water (50 mL), saturated solution of NaHCO₃ (50 mL), and brine (500 mL). The organic solution was dried over sodium sulfate and concentrated. Purification of the residue by flash chromatography using EtOAc/hexane as eluent provided the title compound (0.28 g, 52%) as white foam. R_{f} 0.3 (EtOAc/hexane 3/7); ¹H NMR (CDCl₃) δ0.90-1.00 (m, 6H), 1.43 (s, 9H), 1.60-180 (m, 1H), 1.90-2.06 (m, 2H), 2.70 (t, J=7.6 Hz, 2H), 3.10-3.22 (m, 2H), 4.03-4.22 (m, 4H), 4.65-4.80 (m, 2H), 6.24-6.40 (m, 2H), 6.85-6.95 (m, 1H), 7.02 (t, J=7.2 Hz, 1H), 7.10-7.35 (m, 7H), 8.22 (brs, 1H); MS (m/z): 520 (M+1).

### EXAMPLE 6

### Preparation of N-(1-indolinyl)-(E)-4-((N-(benzylsulfonyl)leucyl)amino)-6-phenyl-2-hexenamide

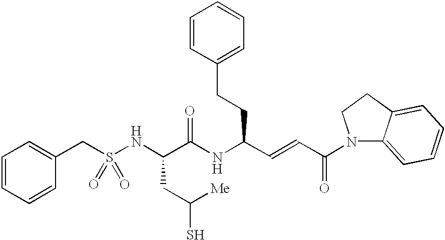

N-(1-Indolinyl)-(E)-4-((N-(tert-butoxycarbonyl)leucyl)amino)-6-phenyl-2-hexenamide (0.19 g, 0.36 mmol) was added to a 4M solution of HCl in dioxane (6 mL). The resulting solution was stirred at 25° C. for 3 h then was concentrated under reduced pressure to afford the amine intermediate as a white solid.

To a mixture of the above intermediate in THF (10 mL) were added α-toluenesulfonyl chloride (0.10 g, 0.53 mmol) and triethylamine (0.20 mL, 1.42 mmol). The resulting mixture was stirred at 25° C. for 18 h then was partitioned between water (50 mL) and EtOAc (50 mL). The organic layer was washed with saturated solution of NH₄Cl (50 mL), brine (500 mL), dried over sodium sulfate and concentrated. Purification of the residue by flash chromatography using EtOAc/hexane as eluent provided the title compound (0.10 g, 50%) as white foam. ¹H NMR (CDCl₃) δ0.80-0.95 (m, 6H), 1.32-1.42 (m, 1H), 1.50-1.70 (m, 2H), 1.90-2.06 (m, 2H), 2.62-2.75 (m, 2H), 3.10-3.20 (m, 2H), 3.70-3.80 (m, 1H), 4.03-4.18 (m, 2H), 4.22-4.30 (m, 2H), 4.65-4.80 (m, 2H), 6.24-6.34 (m, 1H), 6.37-6.45 (m, 1H), 6.85-6.95 (m, 1H), 7.02 (t, J=7.2 Hz, 1H), 7.10-7.45 (m, 15H), 8.20 (brs, 1H); MS (m/z): 574 (M+1).

### EXAMPLE 7

### Preparation of N-(2-phenyl-1-ethyl)-3-((N-benzyloxycarbonyl)phenyl-alaninyl)-2-oxo-4-(4-methoxyphenyl)butanamide

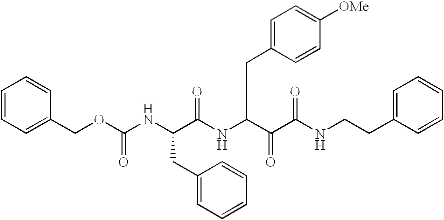

N-(tert-butoxycarbonyl)-O-methyltyrosine (51 mmol, 15 g) was stirred with 1.5 eq. EDC, 1 eq. HOBt in 200 mL anhydrous acetonitrile under nitrogen for 30 min. 2 eq. N,O-dimethylhydroxylamine.HCl was added, followed by 4 eq. NMM (N-methylmorpholine) and the mixture was stirred overnight. TLC of the mixture on silica (GF254) eluted in 5% isopropanol (IPA) in dichloromethane (DCM) showed one spot at the solvent front. The mixture was filtered to remove salts, rotoevaporated and dissolved into 200 mL EtOAc. The solution was washed separately with 100 mL each of 1 N HCl, saturated sodium bicarbonate, distilled water, and brine, then dried over sodium sulfate, filtered, and rotoevaporated. The solid Weinreb amide, N-(tert-butoxycarbonyl)-O-methyltyrosine N-methoxy-N-methylamide, was dissolved in 200 mL toluene, rotoevaporated, and placed under vacuum overnight. Yield 15.3 g, 89% purity by NMR. ¹H-NMR (CDCl₃): s, 9H, 1.3 ppm (Boc); dd, 1H, 2.8 ppm (CbH1); dd, 1H, 2.9 ppm (CbH2); s, 3H, 3.1 ppm(N-Me); s, 3H, 3.7 ppm (Ar-OMe); s, 3H, 3.8 ppm (N-OMe); m, 1H, 4.9 ppm (CaH); m, 1H 5.1 ppm (NH); d, 2H, 6.8 ppm (CdH); d 2H, 7.1 ppm (CeH).

N-(tert-butoxycarbonyl)-O-methyltyrosine N-methoxy-N-methylamide (45 mmol, 15.3 g) was dissolved in 200 mL anhydrous tetrahydrofuran (THF) under nitrogen and cooled to −70° C. 1 eq. lithium aluminum hydride (LAH) in 45 mL THF was added over 15 min., keeping the temperature below −60° C. and reacted at −70° C. for 1 5 min. The reaction was removed from the dry ice bath, allowed to warm to −10° C. over 40 min, and cooled back down to −70° C. 45 mL of 2 M aqueous potassium hydrogen sulfate was added over 2 min, after which the temperature was found to be −10° C. The mixture was allowed to warm up to 0° C. TLC (3% MeOH/DCM) showed that all starting material had been converted to the reduced form which eluted slower than the amide. The mixture was filtered to remove salts, rotoevaporated and dissolved into 200 mL EtOAc. The solution was washed separately with 60 mL each of 1 N HCl, saturated sodium bicarbonate, and brine, then dried over sodium sulfate, filtered, and rotoevaporated. The product was dissolved in 125 mL THF, added dropwise over 10 min. to a solution of 5 eq. potassium cyanide (KCN) and 16 eq. potassium bicarbonate in 125 mL distilled water, and the reaction allowed to proceed for 30 min. at room temperature. The THF layer was separated, rotoevaporated, and the residue dissolved in EtOAc. The solution of the cyanohydrin, 3-(N-(tert-butoxycarbonyl)amino)-2-hydroxy-3-(4-methoxyphenyl)butanonitrile, was washed separately each in 60 mL, distilled water and brine, then dried over sodium sulfate, filtered and rotoevaporated. Yield 12.3 g, 89% pure by RP-HPLC (10-90 gradient of 0.1% TFA in 90% acetonitrile/l0% distilled deionized water over 0.1% TFA in distilled deionized water). ¹H-NMR(CDCl₃): S, 9H, 1.4 ppm (Boc); m, 1H, 2.9 ppm (CbH1); m, 1H, 3.1 ppm (CbH2), s, 3H, 3.8 ppm (Ar-OMe); d, 1H, 4.5 ppm (C1H); m, 1H, 4.8 ppm (CaH); m, 1H, 4.9 ppm (NH); b, 0.5H, 5.4 ppm (OH); d, 2H, 6.8 ppm (CdH); d 2H, 7.1 ppm (CeH).

The product was azeotroped with 100 mL toluene to remove remaining EtOAc, acetonitrile, and MeOH. 3-(N-(tert-butoxycarbonyl)-amino)-2-hydroxy-3-(4-methoxyphenyl)butanonitrile (40 mmol, 12.3 g) was dissolved in 85 mL of 1:1 concentrated HCl/dioxane and heated to reflux for 14 h. RP-HPLC (10-90 gradient) showed absence of starting material. The reaction was rotoevaporated, redissolved into 5 mL MeOH, then precipitated into 100 mL ethyl ether. The deprotected hydroxyacid product, 3-amino-2-hydroxy-3-(4-methoxyphenyl)butanoic acid hydrochloride, was filtered and dried. Yield 4.7 g. ¹H-NMR (CDCl₃): m, 2H, 3.0 ppm (CbH); m, 1H, 3.3 ppm (CaH); s, 3H, 3.8 ppm(Ar-OMe); m, 1H, 4.1 ppm (C1H); d, 2H, 6.9 ppm (CdH); d 2H, 7.2 ppm (CeH).

3-amino-2-hydroxy-3-(4-methoxyphenyl)butanoic acid hydrochloride (18 mmol, 4.7 g) and 1 eq. Z-OSu were dissolved in 4 eq. NMM and 50 mL anhydrous acetonitrile and stirred overnight. The volatile organic components were rotoevaporated and pH of the residue decreased to 7.5 with saturated sodium bicarbonate. The solution was then washed twice with 50 mL of fresh EtOAc. The pH was adjusted to 2 with 1 N HCl and the product extracted with twelve lots of 50 mL EtOAc. The extracts were washed with brine, dried over anhydrous sodium sulfate, filtered, and rotoevaporated. Yield 2.8 g of 3-(N-(benzyloxycarbonyl)amino)-2-hydroxy-3-(4-methoxyphenyl)butanoic acid.

3-(N-(benzyloxycarbonyl)amino)-2-hydroxy-3-(4-methoxyphenyl)-butanoic acid (0.58 mmol, 208 mg) was dissolved with 1.5 eq. EDC and 1 eq. HOBt in 10 mL anhydrous acetonitrile. The mixture was stirred for 30 min. before 2.5 eq. of phenethylamine was added and the reaction stirred overnight. TLC eluted with 5% IPA in DCM and developed with polymolybdic acid showed one spot with R_{f} 0.7 in addition to starting material. The reaction mixture was rotoevaporated and the residue dissolved in 20 mL EtOAc, then separately washed with 10 mL 1N HCl, saturated sodium bicarbonate, distilled water, and brine. The solution was dried over sodium sulfate, filtered, and rotoevaporated. The residue was triturated with 1:1 ethyl ether/hexanes, rotoevaporated and placed under vacuum overnight. Yield 230 mg, purity 86% N-(2-phenyl-1-ethyl)-3-(N-(benzyloxycarbonyl)amino)-2-hydroxy-3-(4-methoxyphenyl)-butanamide.

The last reaction was repeated on higher scale to increase total yield to 2.7 g.

N-(2-phenyl-1-ethyl)-3-(N-(benzyloxycarbonyl)amino)-2-hydroxy-3-(4-methoxyphenyl)butanamide (6.2 mmol, 2.9 g) was dissolved in 20 mL MeOH under nitrogen. 100 mg of palladium oxide, Pd(OH)₂, was added and reacted under balloon-pressure hydrogen for 3 h. TLC (5% IPA/DCM) showed two closely-spaced spots near origin for the N-deprotected product. The catalyst was filtered off over celite and the solution rotoevaporated. Yield 1.87 g of N-(2-phenyl-1-ethyl)-3-amino-2-hydroxy-3-(4-methoxyphenyl)butanamide. The reaction was repeated to increase total yield to 2.7 g.

0.93 g (1.2 eq., 3.1 mmole) N-(benzyloxycarbonyl)phenylalanine was dissolved with 1.7 eq. EDC and 1.2 eq. HOBt in 20 mL anhydrous acetonitrile under nitrogen and stirred for 30 min. N-(2-phenyl-1-ethyl)-3-amino-2-hydroxy-3-(4-methoxyphenyl)butanamide (1.2 g, 2.6 mmole) and 4.8 eq. NMM were added and the reaction allowed to proceed overnight. TLC (5% IPA/DCM) developed with ninhydrin showed no free amine remained. The reaction was rotoevaporated and the residue dissolved in 30 mL EtOAc. The solution was separately washed with 10 mL 1 N HCl, saturated sodium bicarbonate, distilled water, and brine, then dried over sodium sulfate and filtered. The filtrate was rotoevaporated and triturated with ethyl ether. Yield 1.2 g N-(2-phenyl-1-ethyl)-3-((N-benzyloxycarbonyl)phenylalaninyl)amino-2-hydroxy-3-(4-methoxyphenyl)-butanamide.

0.33 mmole (200 mg) of the hydroxyamide, N-(2-phenyl-1-ethyl)-3-((N-benzyloxycarbonyl)phenylalaninyl)amino-2-hydroxy-3-(4-methoxyphenyl)butanamide, was dissolved in 2 mL DMSO and 2 mL toluene. 10 eq. EDC was added and the solution cooled to 0° C. 5 eq. of DCA was added, the mixture removed from the ice bath, and stirred for 15 min. The reaction was poured into 20 mL distilled water, extracted into 20 mL EtOAc. The organic phase was then washed with 10 mL each of saturated sodium bicarbonate, distilled water, and brine. The solution was dried over sodium sulfate, filtered, and retoevaporated. The product was purified with a silica column eluted with a gradient of 0-2% MeOH in DCM. The fractions were pooled and the residue after rotoevaporation was triturated with acetonitrile/ethyl ether/hexanes. Yield 1 20 mg.

### EXAMPLE 8

### Synthesis of (S)-2-(N-(Morpholinocarbonyl)leucyl)amino-4-phenylbutanal

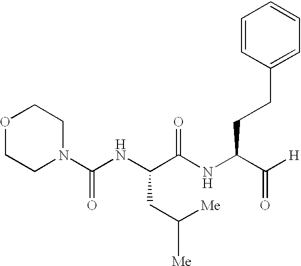

### Step A: Methyl (S)-2-isocyano-4-methylpentenoate

To a solution of leucine methyl ester hydrochloride in DCM was added pyridine (8.9 mL, 110 mmol) and the mixture was cooled to 0° C. A 20% phosgene in toluene solution was added and the mixture was stirred for 2 hours then rotovaped to a colorless oil. Purification was done by Kugelrohr distillation collecting material that distilled 72-80° C. yielding 3.51 g colorless oil (74% yield). ¹H NMR (CDCl₃): 4.0-4.05 ppm (m, 1H); 3.8 ppm (s, 3H); 1.8-1.85 ppm (m, 1 H); 1.6-1.7 ppm (m, 2H); 0.9-0.97 ppm (m, 6H).

### Step B: N-(Morpholinocarbonyl)leucine methyl ester

Methyl (S)-2-isocyano-4-methylpentenoate was dissolved in morpholine (0.6 M, 20 eq.) and stirred for 18 hrs. The resulting mixture was pumped down and purified on 4″×50 mm silica flash column eluting with 4:1 EtOAc:Hexanes. Yield 0.684 g (86%). R_{f}=0.3 in 4/1 EtOAc/Hexanes. ¹H NMR (CDCl₃): 4.8-4.85 ppm (m, 1H); 4.5-4.55 ppm (m, 1H): 3.75 ppm (s, 3H); 3.65-3.67 ppm (m, 4H); 3.35-3.4 ppm (m, 4H); 1.6-1.7 ppm (m, 2H); 1.5-1.58 ppm (m, 1H); 0.95-0.98 ppm (m, 6H).

### Step C: N-(Morpholinocarbonyl)leucine

N-(Morpholinocarbonyl)leucine methyl ester was dissolved in MeOH to make a 0.15 M solution. LiOH solution (1 M in H₂O, 2.2 eq.) was added. After 18 h, no starting material was observed by TLC in 9:1 DCM:MeOH. The crude was poured over 10 mL Dowex and eluted with 1:1 MeOH:H2O (100 mL). The eluant was pumped down to a quantitive yield of product. HPLC: R_{f}=9.1 min in a 5% to 75% acetonitrile gradient in 0.1% aqueous TFA buffer on a 4.6×250 mm, 5 uM particle, 100 angstrom pore, C18 pore, C18 column at a 1 mL/min flow rate. ¹H NMR (CDCl₃): 4.8-4.85 ppm (m, 1H); 4.4-4.45 ppm (m, 1H); 3.7-3.75 ppm (m, 4H); 3.3-3.45 ppm (m, 4H); 1.75-1.8 ppm (m, 2H); 1.55-1.62 ppm (m, 1H); 0.95-0.98 ppm (m, 6H).

### Step D: N-Methoxy-N-methyl-2-( N²-(tert-butoxycarbonyl)amino)-4-phenylbutanamide

A solution of 2-(N-(tert-butoxycarbonyl)amino-4-phenylbutanoic acid (2 g, 7.16 mmol), HCl-N-Methyl-O-Methyl hydroxylamine (1.40 g, 14.32 mmol), EDC (2.74 g, 14.32 mmol), and HOBt (1.10 g, 7.16 mmol) in MeCN (28.6 mL) was stirred for 15 min at room temp. DIEA (6.24 mL, 35.8 mmol) was added and the reaction stirred for 18 hours. The solvent was removed under reduced pressure and the resulting residue re-suspended in ethyl acetate (200 mL) and 1M HCl (20 mL). The ethyl acetate layer was washed with 0.5M HCl (20 mL), saturated NaHCO₃ (2×20 mL), and brine (20 mL). The ethyl acetate was dried with sodium sulfate and solvent removed under reduced pressure, resulting in a quantitative yield of title compound. HPLC: R_{f}=16.0 min in a 5% to 75% acetonitrile gradient in 0.1% aqueous TFA buffer on a 4.6×250 mm, 5 uM particle, 100 angstrom pore, C18 column at a 1 mL/min flow rate. TLC: R_{f}=0.2 in 4/1 EtOAc/Hexanes. ¹H NMR (CDCl₃): 7.15-7.3 ppm (m, 5H); 5.2-5.3 ppm (m, 1H); 4.65-4.75 ppm (m, 1H); 3.6 ppm (s, 3H); 3:15-3.2 ppm (m, 3H); 2.6-2.7 ppm (m, 2H); 2.0-2.1 ppm (m, 1H); 1.75-1.9 ppm (m, 1 H); 1.4 ppm (s, 9H).

### Step E: N-Methoxy-N-methyl-2-amino-4-phenylbutanamide

To N-methoxy-N-methyl-2-(N²-(tert-butoxycarbonyl)amino)-4-phenylbutanamide (2.31 g, 7.16 mmol) was added 40 mL of 6 M HCl in ethanol. The mixture was stirred for 45 minutes, at which time no more starting material was observed by TLC in 4/1 EtOAc/Hexanes. Remove solvent under reduced pressure, the resulting residue was re-suspended in MeCN and the solvent was removed under reduced pressure resulting in a white foam in a quantitative yield. ¹H NMR (D₂O ): 7.1-7.3 ppm (m, 5H); 4.2-4.3 ppm (m, 1H); 3.45 ppm (s, 3H); 3:05 ppm (s, 3H); 2.6-2.75 ppm (m, 2H); 2.05-2.15 ppm (m, 2H).

### Step F: N-Methoxy-N-methyl-2-(N-(morpholinocarbonylleucyl)amino-4-phenylbutanamide

A solution of N-(morpholinocarbonyl)leucine (0.81 g, 3.31 mmol), N-methoxy-N-methyl-2-amino-4-phenylbutanamide (1.03 g, 3.97 mmol), EDC (0.95 g, 4.97 mmol), and HOBt (0.51 g, 3.31 mmol) in MeCN (13.2 mL) was stirred for 15 min at room temp. DIEA (2.88 mL, 16.55 mmol) was added and the reaction stirred for 18 hours. The solvent was removed under reduced pressure and the resulting residue re-suspended in ethyl acetate (200 mL) and 1M HCl (20 mL). The ethyl acetate layer was washed with 0.5 M HCl (20 mL), saturated NaHCO₃ (2×20 mL), and brine (20 mL). The ethyl acetate was dried with sodium sulfate and solvent removed under reduced pressure, resulting in a quantitative yield of title compound. HPLC: R_{f}=15.0 min in a 5% to 75% acetonitrile gradient in 0.1% aqueous TFA buffer on a 4.6×250 mm, 5 uM particle, 100 angstrom pore, C18 column at a 1 mL/min flow rate. ¹H NMR (CDCl₃): 7.15-7.3 ppm (m, 5H); 6.6-6.65 ppm (m, 1H); 4.9-5.0 ppm (m, 2H); 4.35-4.45 ppm (m, 1H), 3.6-3.7 ppm (m, 7H); 3.3-3.4 ppm (m, 4H); 3.15 ppm (s, 3H); 2.6-2.75 ppm (m, 2H); 1.9-2.1 ppm (m, 2H); 1.5-1.7 ppm (m, 3H); 0.95-1.0 ppm (m, 6H).

### Step G: (S)-2-(N-(Morpholinocarbonylleucyl)amino)-4-phenylbutanal

To a solution of N-methoxy-N-methyl-2-(N-(morpholinocarbonylleucyl)amino-4-phenylbutanamide (1.93 g, 4.31 mmol) in THF cooled to −78° C. was added LAH (4.31 mL of 1 M in THF, 4.31 mmol) dropwise over 20 min. The reaction was then warmed to 0° C. for 30 minutes at which time there was no more starting material by TLC in 4/1 EtOAc/Hexanes. The reaction was again cooled to −78° C. and a 2 M solution of KHSO₄ (17.23 mL, 8.61 mmol) was added dropwise. The reaction was poured into 800 mL of EtOAc and was washed with 0.5M HCl (2×80 mL), saturated NaHCO₃ (2×80 mL), and brine (80 mL). The ethyl acetate was dried with sodium sulfate and solvent removed under reduced pressure, resulting in a white foam. The residue is then loaded directly onto a 22×250 mm, 10-15 uM particle, 100 angstrom pore, C18 column at a 25 mL/min flow rate and was eluted with a 10-50% MeCN in 10 mM ammonium acetate buffer (pH˜6.5). Product containing fractions were pooled and lyophilized yielding 1.3 g (77.5% of the title compound as a white powder. HPLC: R_{f}=13.5 min in a 5% to 75% acetonitrile gradient in 0.1% aqueous TFA buffer on a 4.6×250 mm, 5 uM particle, 100 angstrom pore, C18 column at a 1 mL/min flow rate. ¹H NMR (CD₃OD): 7.1-7.3 ppm (m, 5H); 4.4-4.45 ppm (m, 1H); 4.25-4.35 ppm (m, 1H); 3.8-3.9 ppm (m, 1H); 3.55-3.7 (m, 4H); 3.3-3.45 ppm (m, 4H); 2.45-2.7 (m, 2H); 1.9-2.05 ppm (m, 1H); 1.55-1.8 ppm (m, 4H); 0.9-1.0 ppm (m, 6H).

### EXAMPLE 9

Other compounds that have been prepared by the above methods, or routine modifications thereof, include, but are not limited to: N-(N-(4-methylpiperazinylcarbonyl)leucyl)tyrosinal, (S)-2-(N-(4-methylpiperazinylcarbonyl)leucyl)amino-4-phenylbutanal, N-(N-(morpholinocarbonyl)leucyl)tyrosinal, N-(N-(benzyloxycarbonyl)leucyl)-tyrosinal, (S)-2-(N-(phenylsulfonyl)leucyl)amino-4-phenylbutanal, N-(N-(phenylsulfonyl)leucyl)tyrosinal, (S)-2-(N-(morpholinocarbonyl)phenylalanyl)amino-4-phenylbutanal, N-(N-(benzyloxycarbonyl)phenylalanyl)-tyrosinal, (S)-2-(N-(benzyloxycarbonyl)phenylalanyl)amino-4-phenylbutanal, (S)-2-(N-(phenylsulfonyl)phenylalanyl)amino-4-phenylbutanal, N-(N-(morpholinocarbonyl)phenylalanyl)tyrosinal, N-(N-(4-methylpiperazinylcarbonyl)phenylalanyl)tyrosinal, (S)-2-(N-(4-methylpiperazinylcarbonyl)phenylalanyl)amino-4-phenylbutanal, N-(N-(phenylsulfonyl)phenylalanyl)tyrosinal, (S)-2-(N-(benzyloxycarbonyl)-leucyl)amino-4-phenylbutanal, N-(2-phenyl-1-carbamoyl-1-ethyl)-3-((N-benzyloxycarbonyl)phenylalaninyl)-2-oxo-5-phenylpentanamide, N-(2-(2-pyridyl)-1-ethyl)-3-((N-benzyloxycarbonyl)phenylalaninyl)-2-oxo-4-(4-methoxyphenyl)butanamide, N-(2-(2-pyridyl)-1-ethyl)-3-((N-benzyloxycarbonyl)leucyl)-2-oxo-4-(4-methoxyphenyl)butanamide, N-(2,2-diphenyl-1-ethyl)-3-((N-benzyloxycarbonyl)phenylalaninyl)-2-oxo-4-(4-methoxyphenyl)butanamide, N-(2-(1-methyl-3-indolyl)-1-ethyl)-3-((N-benzyloxycarbonyl)leucyl)-2-oxo-4-(4-methoxyphenyl)butanamide, N-(2-(1-benzyl-3-indolyl)-1-ethyl)-3-((N-benzyloxycarbonyl) leucyl)-2-oxo-4-(4-methoxyphenyl)butanamide, N-(2-phenyl-1-carbamoyl-1-ethyl)-3-((N-morpholinocarbonyl)leucyl)-2-oxo-5-phenylpentanamide, N-(2-phenyl-1-ethyl)-3-((N-benzyloxycarbonyl)leucyl)-2-oxo-4-(4-methoxyphenyl)butanamide, N-(2-phenyl-1-ethyl)-3-((N-benzyloxycarbonyl)phenylalaninyl)-2-oxo-4-(4-methoxyphenyl)butanamide, N-(2-phenyl-1-ethyl)-3-((N-phenylsulfonyl)leucyl)-2-oxo-4-(4-methoxyphenyl)butanamide, N-(2-phenyl-1-ethyl)-3-((N-phenylsulfonyl)phenylalaninyl)-2-oxo-4-(4-methoxyphenyl)butanamide and N-(1-indolinyl)-(E)-4-((N-(benzylsulfonyl)-phenylalaninyl)amino)-6-phenyl-2-hexenamide.

### EXAMPLE 10

### Assays for Falcipain Inhibition

Compounds provided herein for use in the compositions and methods can be and have been tested for modulation of falcipain activity, particularly inhibition of falcipain, in assays known to those of skill in the art. See, e.g., Rosenthal et al. (1996) Antimicrob. Agents Chemother. 40(7):1600-1603; Dominguez et al. (1997) J. Med. Chem. 40:2726-2732; Clark et al. (1994) Molec. Biochem. Parasitol. 17:129; Ring et al. (1993) Proc. Natl. Acad. Sci. USA 90:3583-3587.

Assays of the hydrolysis of the fluorogenic substrate benzyloxy-carbonyl-Phe-Arg-7-amino-4-methylcoumarin (Z-Phe-Arg-AMC) were performed with a 96 well format for spectrofluorometry. See, e.g., Rosenthal et al. (1989) Mol. Biochem. Parasitol. 35:177-184. P. falciparum trophozoite extracts containing falcipain were prepared as described in Rosenthal et al. (1993) J. Clin. Invest. 91:1052-1056. For each of multiple experiments, extracts (in 0.1 M sodium acetate and 10 mM dithiothreitol (pH 5.5)) containing identical concentrations of enzyme (˜30 nM; calculated by titration with the stoichiometric cysteine proteinase inhibitor l-trans-epoxy-succinyl-leucylamido-(4-guanidino)-butane (E-64, Sigma)) were incubated with each compound provided herein (added from 100×stocks in dimethyl sulfoxide (DMSO)) for 30 min at room temperature before the substrate was added. Fluorescence caused by the cleavage of Z-Phe-Arg-AMC was monitored continuously over 30 min at room temperature with a Labsystems Fluoroskan II spectrofluorometer.

### Results

The rate of hydrolysis of Z-Phe-Arg-AMC (increase in fluorescence/time) in the presence of a compound provided herein was compared with the rate of hydrolysis in negative controls incubated with an equivalent volume of DMSO and with positive controls incubated with E-64 (10 μM).

The IC₅₀ for falcipain inhibitory activity for each of the compounds specifically disclosed herein has been measured. Almost all of the compounds have an IC₅₀ of less than 100 nM. Many of the compounds have an IC₅₀ less than about 50 nM, and some of the compounds have an IC₅₀ less than about 10 nM.

### EXAMPLE 11

### Assays for Cruzain Inhibition

Compounds provided herein for use in the compositions and methods can be and have been tested for modulation of cruzain activity, particularly inhibition of cruzain, in assays known to those of skill in the art. See, e.g., Engel et al. (1 998) J. Exp. Med. 188(4):725-734; Li et al. (1995) J. Med. Chem. 38:5031.

Assays for cruzain inhibition were performed similarly to those for falcipain inhibition (see, Example 10) using recombinant cruzain prepared according to Eakin et al. (1992) J. Biol. Chem. 267(11):7411-7420. Briefly, a 1438-bp fragment of a DNA predicted to encode the proform of cruzain (from Cys⁻¹⁰⁴ to 100 bp downstream of the stop codon) was amplified with the polymerase chain reaction. The oligonucleotides used in this amplification added a 5′ Xhol site, upstream DNA sequences encoding an enteropeptidase cleavage site, and an Xbal site at the 3′ end of the gene. The expression plasmid, pCheY15LOX (Sigal et al. (1990) J. Biol. Chem. 265:5113-5120), was digested with SaA and Sbal to remove the lipoxygenase gene. Since Xhol and SaA generate compatible sticky ends, the Shol-Xbal PCR-amplified fragment was ligated into the plasmid (pCheYTc) to permit expression of the proform of the protease as a fusion with the E. coli CheY protein under control of the lac promotor.

Cultures of E. coli (strain DH5a containing the expression plasmid) were grown overnight, diluted 10-fold into fresh LB medium plus 100 μg/ml ampicillin, and allowed to recover at 37° C. for 1 h. IPTG was added to 1 mM, and the cultures were induced at 37° C. with shaking for 4 h. Cell lysis, urea solubilization, and refolding were performed as described by Marston et al. (1984) Bio/Technology 2:800-804 with the following exceptions. Insoluble proteins were solubilized in 7 M urea, and after a pH 10.7 refolding step and subsequent incubation at pH 8.0, the soluble proteins were precipitated with ammonium sulfate at 40% saturation. The precipitated proteins were collected by centrifugation and resuspended in 0.1 M sodium acetate, pH 5.5, and dialyzed against two changes of 10-fold excess of the same sodium acetate buffer to remove other salts. The proteins were then fractionated by ion exchange chromatography on DEAE-Sepharose using a 0-1 M gradient of NaCl.

### Results

The rate of hydrolysis of Z-Phe-Arg-AMC (increase in fluorescence/time) by the recombinant cruzain in the presence of a compound provided herein was compared with the rate of hydrolysis in negative controls incubated with an equivalent volume of DMSO and with positive controls incubated with E-64 (10 μM). See, Example 10.

The IC₅₀ for cruzain inhibitory activity for each of the compounds specifically disclosed herein has been measured. Almost all of the compounds have an IC₅₀ of less than 100 nM. Many of the compounds have an IC₅₀ less than about 50 nM, and some of the compounds have an IC₅₀ less than about 10 nM.

Since modifications will be apparent to those of skill in this art, it is intended that this invention be limited only by the scope of the appended claims.

What is claimed is:

1. A compound of formula II: 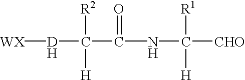
or a pharmaceutically acceptable derivative thereof, wherein:
D is nitrogen;
R² is alkyl, alkenyl, alkynyl, aralkyl, heteroaralkyl, -alkylsulfurylalkyl, -alkenylsulfurylalkenyl or -alkynylsulfurylalkynyl; and
R¹, W and X are selected from (i), (ii) or (iii) as follows:
(i) R¹ is aralkyl or heteroaralkyl, with the proviso that R¹ is not 3-indolylmethyl;
W is heteroaryl or heterocyclyl; and
X is —C(O)—; or
(ii) R¹ is aralkyl or heteroalkaryl;
W is alkyl, alkenyl, alkynyl, aralkyl, heteroaralkyl, aryl, heteroaryl, bicyclic alkyl or heterocyclyl; and
X is —SOₙ— where n is an integer from 0 to 2;
with the provisos that (i) if X is SO₂, then R¹ is not subsituted or unsubstituted benzyl or CH₂heteroaryl; and (ii) if R¹ is —CH₂-(para-hydroxy)phenyl or —CH₂-(para-isopropoxy)-phenyl, then W is not naphthyl; or
(iii) R¹ is aralkyl or heteroalkaryl;
W is alkyl, alkenyl, alkynyl, aralkyl, heteroaralkyl, aryl, heteroaryl, bicyclic alkyl or heterocyclyl; and
X is —OC(O)—;
with the provisos that (i) the alkyl portion of R¹ has 2 to 6 carbons; and (ii) if the alkyl portion of R¹ is ethylene, then R² is not isopropyl or benzyl.

2. The compound of claim 1, wherein the alkyl portion of R¹ has from about 2 to about 6 carbons.

3. The compound of claim 1, wherein R¹, W and X are selected as in (i) only.

4. The compound of claim 1, wherein R¹, W and X are selected as in (ii) only.

5. The compound of claim 1, wherein R¹, W and X are selected as in (iii) only.

6. The compound of claim 1, wherein R² is isobutyl, benzyl or —CH₂CH₂SO₂CH₃.

7. The compound of claim 3, wherein W is 4-methylpiperazinyl or morpholino.

8. The compound of claim 3, wherein R¹ is 4-hydroxybenzyl or 2-phenyl-1-ethyl.

9. The compound of claim 4, wherein W is aryl, heteroaryl or heterocyclyl.

10. The compound of claim 9, wherein W is phenyl.

11. The compound of claim 4, wherein R¹ is 4-hydroxybenzyl or 2-phenyl-1-ethyl.

12. The compound of claim 5, wherein W is alkyl, aralkyl, aryl or bicyclic alkyl.

13. The compound of claim 12, wherein W is benzyl.

14. The compound of claim 5, wherein R¹ is 4-hydroxybenzyl or 2-phenyl-1-ethyl.

15. The compound of claim 1 selected from the group consisting of N-(N-(4-methylpiperazinylcarbonyl)leucyl)tyrosinal, (S)-2-(N-(4-methylpiperazinylcarbonyl)leucyl)amino-4-phenylbutanal, N-(N-(morpholinocarbonyl)leucyl)tyrosinal, (S)-2-(N-(phenylsulfonyl)-leucyl)amino-4-phenylbutanal, N-(N-(phenylsulfonyl)leucyl)tyrosinal, (S)-2-(N-(morpholinocarbonyl)phenylalanyl)amino-4-phenylbutanal, (S)-2-(N-(phenylsulfonyl)phenylalanyl)amino-4-phenylbutanal, N-(N-(morpholino-carbonyl)phenylalanyl)tyrosinal, N-(N-(4-methylpiperazinylcarbonyl)phenylalanyl)tyrosinal, (S)-2-(N-(4-methylpiperazinylcarbonyl)phenylalanyl)amino-4-phenylbutanal, N-(N-(phenylsulfonyl)phenylalanyl)tyrosinal, (S)-2-(N-(benzyloxycarbonyl)leucyl)amino-4-phenylbutanal and (S)-2-(N-(morpholinocarbonyl)leucyl)amino-4-phenylbutanal.

16. The compound of claim 1 selected from the group consisting of (S)-2-(N-(4-methylpiperazinylcarbonyl)leucyl)amino-4-phenylbutanal, (S)-2-(N-(phenylsulfonyl)leucyl)amino-4-phenylbutanal, (S)-2-(N-(morpholinocarbonyl)phenylalaninyl)amino-4-phenylbutanal, (S)-2-(N-(phenylsulfonyl)phenylalaninyl)amino-4-phenylbutanal, (S)-2-(N-(4-methylpiperazinylcarbonyl)phenylalaninyl)amino-4-phenylbutanal, (S)-2-(N-(benzyloxycarbonyl)leucyl)amino-4-phenylbutanal and (S)-2-(N-(morpholinocarbonyl)leucyl)amino-4-phenylbutanal.

17. The compound of claim 1 selected from the group consisting of (S)-2-(N-(4-methylpiperazinylcarbonyl)leucyl)amino-4-phenylbutanal, (S)-2-(N-(phenylsulfonyl)leucyl)amino-4-phenylbutanal, (S)-2-(N-(morpholinocarbonyl)phenylalaninyl)amino-4-phenylbutanal, (S)-2-(N-(phenylsulfonyl)phenylalaninyl)amino-4-phenylbutanal, (S)-2-(N-(4-methylpiperazinylcarbonyl)phenylalaninyl)amino-4-phenylbutanal and (S)-2-(N-(morpholinocarbonyl)leucyl)amino-4-phenylbutanal.

18. A compound of formula III: 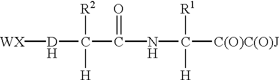
or a pharmaceutically acceptable dervative thereof, wherein:
W is hydrogen, alkyl, alkenyl, alkynyl, aralkyl, heteroaralkyl, aryl, heteroaryl, bicyclic alkyl or heterocyclyl;
X is a direct link, —C(O)—, —OC(O)— or —SOₙ— where n is an integer from 0 to 2, preferably 2;
D is nitrogen;
R² is alkylalkenyl, alkynyl, aralkyl, heteroaralkyl, -alkylsulfurylalkyl, -alkenylsulfurylalkenyl or -alkynylsulfurylalkynyl;
R¹ is selected from among (i), (ii) or (iii) as follows:
(i) aryl or heteroaryl;
(ii) aralkyl where (a) the alkyl portion has one carbon atom and the aryl portion is substituted with at least one non-hydrogen substituent, or (b) the alkyl portion has at least two carbon atoms; or
(iii) heteroaralkyl; and
J is -alkylcarbamoyl, -alkenylcarbamoyl, -alkynylcarbamoyl, -alkylamide, -alkenylamide, -alkynylamide, substituted or unsubstituted aryl, substituted or unsubstituted aralkyl, substituted or unsubstituted heteroaryl, or substituted or unsubstituted heteroaralkyl.

19. The compound of claim 18, wherein R¹ is selected as in (i) only.

20. The compound of claim 18, wherein R¹ is selected as in (ii) only.

21. The compound of claim 18, wherein R¹ is selected as in (iii) only.

22. The compound of claim 18, wherein:
W is hydrogen, C₁₋₄alkyl, benzyl, phenyl, camphoryl, C₁₋₄alkylpiperazinyl or morpholino;
X is a direct link, —C(O)—, —OC(O)— or —SO₂—;
D is nitrogen;
R² is isobutyl or benzyl, or is —CH₂CH₂SO₂CH₃;
R¹ is 4-hydroxybenzyl, 2-phenyleth-1-yl or 4-methoxybenzyl; and
J is —CH(CH₂Ph)(CONH₂), —CH₂CH₂-(2-pyridyl), —CH₂CH₂Ph, —CH₂CHPh₂, —CH₂CH₂-(1-methyl-3-indolyl) or —CH₂CH₂-(1-benzyl-3-indolyl).

23. The compound of claim 22, where R¹ is 2-phenyleth-1-yl or 4-methoxybenzyl.

24. The compound of claim 18, wherein:
W is benzyl, phenyl or morpholino;
X is a direct link, —C(O)—, —OC(O)— or —SO₂—;
D is nitrogen;
R² is isobutyl or benzyl;
R¹ is 2-phenyleth-1-yl or 4-methoxybenzyl; and
J is —CH(CH₂Ph)(CONH₂), —CH₂CH₂-(2-pyridyl), —CH₂CH₂Ph, —CH₂CHPh₂, —CH₂CH₂-(1-methyl-3-indolyl) or —CH₂CH₂-(1-benzyl-3-indolyl).

25. The compound of claim 18 that is selected from the group consisting of N-(2-phenyl-1-carbamoyl-1-ethyl)-3-((N-benzyloxycarbonyl)-phenylalaninyl)-2-oxo-5-phenylpentanamide, N-(2-(2-pyridyl)-1-ethyl)-3-((N-benzyloxycarbonyl)phenylalaninyl)-2-oxo-4-(4-methoxyphenyl)butanamide, N-(2-phenyl-1-ethyl)-3-((N-benzyloxycarbonyl)cyclohexylalaninyl)-2-oxo-4-(4-methoxyphenyl)butanamide, N-(2-(2-pyridyl)-1-ethyl)-3-((N-benzyloxycarbonyl)leucyl)-2-oxo-4-(4-methoxyphenyl)butanamide, N-(2,2-diphenyl-1-ethyl)-3-((N-benzyloxycarbonyl)phenylalaninyl)-2-oxo-4-(4-methoxyphenyl)butanamide, N-(2-(1-methyl-3-indolyl)-1-ethyl)-3-((N-benzyloxycarbonyl)leucyl)-2-oxo-4-(4-methoxyphenyl)butanamide, N-(2-(1-benzyl-3-indolyl)- 1-ethyl)-3-((N-benzyloxycarbonyl)leucyl)-2-oxo-4-(4-methoxyphenyl)butanamide, N-(2-phenyl- 1-carbamoyl-1-ethyl)-3-((N-morpholinocarbonyl)leucyl)-2-oxo-5-phenylpentanamide, N-(2-phenyl-1-ethyl)-3-((N-benzyloxycarbonyl)leucyl) -2-oxo-4-(4-methoxyphenyl)-butanamide, N-(2-phenyl-1-ethyl)-3-((N-benzyloxycarbonyl)phenylalaninyl)-2-oxo-4-(4-methoxyphenyl)butanamide, N-(2-phenyl-1-ethyl)-3-((N-phenylsulfonyl)leucyl)-2-oxo-4-(4-methoxyphenyl)butanamide and N-(2-phenyl-1-ethyl)-3-((N-phenylsulfonyl)phenylalaninyl)-2-oxo-4-(4-methoxyphenyl)butanamide.

26. A compound of formula IV: 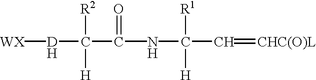
or a pharmaceutically acceptable derivative thereof, wherein:
W is hydrogen, alkyl, alkenyl, alkynyl, aralkyl, heteroaralkyl, aryl, heteroaryl, bicyclic alkyl or heterocyclyl;
X is a direct link, —C(O)—, —OC(O)— or —SOₙ— where n is an integer from 0 to 2, preferably 2;
D is nitrogen;
R² is alkyl, alkenyl, alkynyl, aralkyl, heteroaralkyl, -alkylsulfurylalkyl, -alkenylsulfurylalkenyl or -alkynylsulfurylalkynyl;
R¹ is selected from among (i) or (ii) as follows:
(i) aryl, heteroaryl or heteroaralkyl; or
(ii) aralkyl where (a) the alkyl portion has at least two carbon atoms, or (b) the alkyl portion has one carbon atom and R² is not benzyl, 3-indolylmethyl or isopropyl; and
L is oxaalkyl, oxaalkenyl, oxaalkynyl, alkylamino, arylamino, dialkylamino, (alkyl)(aryl)amino, diarylamino, heteroarylamino, diheteroarylamino, (alkyl)(heteroaryl)amino, (aryl)(heteroaryl)amino, amino, heteroaryl or heterocyclyl.

27. The compound of claim 26, wherein L is heteroaryl or heterocyclyl.

28. The compound of claim 26, wherein R¹ is selected as in (i) only.

29. The compound of claim 26, wherein R¹ is selected as in (ii) only.

30. The compound of claim 26, wherein:
W is hydrogen, C₁₋₄alkyl, benzyl, phenyl, camphoryl, C₁₋₄alkylpiperazinyl or morpholino;
X is a direct link, —C(O)—, —OC(O)— or —SO₂—;
D is nitrogen;
R² is isobutyl or benzyl, or is —CH₂CH₂SO₂CH₃;
R¹ is 4-hydroxybenzyl, benzyl, 2-phenyleth-1-yl or 4-methoxybenzyl; and
L is oxaalkyl or heterocyclyl.

31. The compound of claim 26, wherein L is heterocyclyl.

32. The compound of claim 26, wherein:
W is C₁₋₄alkyl or benzyl;
X is —OC(O)— or —SO₂—;
D is nitrogen;
R² is isobutyl or benzyl, or is —CH₂CH₂SO₂CH₃;
R¹ is benzyl or 2-phenyleth-1-yl; and
L is methoxy or 1-indolinyl.

33. The compound of claim 26, wherein W is tert-butyl or benzyl and L is 1-indolinyl.

34. The compound of claim 26 selected from the group consisting of methyl (E)-4-((N-(benzyloxycarbonyl)phenylalanyl)amino)-5-phenyl-2-pentenoate, methyl (E)-4-((N-(benzyloxycarbonyl)phenylalanyl)-amino)-6-phenyl-2-hexenoate, N-(1-indolinyl)-(E)-4-((N-(tert-butoxy-carbonyl)phenylalanyl)amino)-6-phenyl-2-hexenamide, N-(1-indolinyl)-(E)-4-((N-(tert-butoxycarbonyl)leucyl)amino)-6-phenyl-2-hexenamide, N-(1-indolinyl)-(E)-4-((N-tert-butoxycarbonyl-S,S-dioxomethioninyl)amino)-6-phenyl-2-hexenoate, N-(1-indolinyl)-(E)-4-((N-(benzylsulfonyl)phenylalanyl)-amino)-6-phenyl-2-hexenamide and N-(1-indolinyl)-(E)-4-((N-(benzyl-sulfonyl)leucyl)amino)-6-phenyl-2-hexenamide.

35. A pharmaceutical composition, comprising therapeutially effective amount of a compound of claim 1 in a pharmaceutically acceptable carrier.

36. A pharmaceutical composition, comprising a therapeutically effective amount of a compound of claim 18 in a pharmaceutically acceptable carrier.

37. A pharmaceutical composition, comprising a therapeutically effective amount of a compound of claim 26 in a pharmaceutically acceptable carrier.

38. An article of manufacture, comprising packaging material, a compound of claim 1 or a pharmaceutically acceptable derivative thereof, which is effective for inhibiting falcipain or cruzain, or for treatment, prevention or amelioration of one or more symptoms of parasitic infections, and a label that indicates that the compound or pharmaceutically acceptable derivative thereof is used for inhibiting falcipain or cruzain, or for treatment, prevention or amelioration of one or more symptoms of parasitic infections.

39. An article of manufacture, comprising packaging material, a compound of claim 18 or a pharmaceutically acceptable derivative thereof, which is effective for inhibiting falcipain or cruzain, or for treatment, prevention or amelioration of one or more symptoms of parasitic infections, and a label that indicates that the compound or pharmaceutically acceptable derivative thereof is used for inhibiting falcipain or cruzain, or for treatment, prevention or amelioration of one or more symptoms of parasitic infections.

40. An article of manufacture, comprising packaging material, a compound of claim 26 or a pharmaceutically acceptable derivative thereof, which is effective for inhibiting falcipain or cruzain, or for treatment, prevention or amelioration of one or more symptoms of parasitic infections, and a label that indicates that the compound or pharmaceutically acceptable derivative thereof is used for inhibiting falcipain or cruzain, or for treatment, prevention or amelioration of one or more symptoms of parasitic infections.

41. A method of inhibiting falcipain, comprising administering a therapeutically effective amount of a compound of claim 1.

42. A method of inhibiting falcipain, comprising administering a therapeutically effective amount of a compound of claim 18.

43. A method of inhibiting falcipain, comprising administering a therapeutically effective amount of a compound of claim 26.

44. A method of inhibiting cruzain, comprising administering a therapeutically effective amount of a compound of claim 1.

45. A method of inhibiting cruzain, comprising administering a therapeutically effective amount of a compound of claim 18.

46. A method of inhibiting cruzain, comprising administering a therapeutically effective amount of a compound of claim 26.

47. A method of treating, preventing, or ameliorating one or more symptoms of parasitic infection, comprising administering a therapeutically effective amount of a compound of claim 1.

48. A method of treating, preventing, or ameliorating one or more symptoms of parasitic infection, comprising administering a therapeutically effective amount of a compound of claim 18.

49. A method of treating, preventing, or ameliorating one or more symptoms of parasitic infection, comprising administering a therapeutically effective amount of a compound of claim 26.

50. A method of inhibiting the development or growth of mammalian parasites, comprising administering a therapeutically effective amount of a compound of claim 1.

51. A method of inhibiting the development or growth of mammalian parasites, comprising administering a therapeutically effective amount of a compound of claim 18.

52. A method of inhibiting the development or growth of mammalian parasites, comprising administering a therapeutically effective amount of a compound of claim 26.

53. A method of inhibiting falcipain, comprising administering a therapeutically effective amount of a compound of formula I: 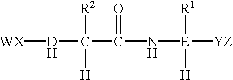
or a pharmaceutically acceptable derivative thereof, wherein:
W is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, unsubstituted or substituted heteroaralkyl, unsubstituted or substituted alkylheteroaryl, unsubstituted or substituted heterocyclyl, or unsubstituted or substituted bicycloalkyl, bicycloalkenyl or bicycloalkynyl;
X is a direct link, —C(O)—, —OC(O)— or —S(O)ₙ— where n is an integer from 0 to 2;
D is nitrogen;
R² is selected from among H, alkyl, alkenyl, alkynyl, oxaalkyl, oxaalkenyl, oxaalkynyl, hydroxyalkyl, hydroxyalkenyl, hydroxyalkynyl, -alkylcarbonylalkyl, -alkenylcarbonylalkenyl, -alkynylcarbonylalkynyl, -alkylamine, -alkenylamine, -alkynylamine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylsulfurylalkyl, -alkenylsulfurylalkenyl, -alkynylsulfurylalkynyl, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, and unsubstituted or substituted heteroaralkyl;
E is carbon;
R¹ is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, or unsubstituted or substituted heteroaralkyl;
Y is —C(O)—, —A′H═CHC(O)— or —A′(O)C(O)NH— where A′ is carbon; and Z is G, J or L where G is hydrogen; and J and L are each independently selected from among H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, alkylamino, arylamino, dialkylamino, (alkyl)(aryl)amino, diarylamino, heteroarylamino, diheteroarylamino, (alkyl)(heteroaryl)amino, (aryl)(heteroaryl)amino, amino, heteroaryl or heterocyclyl;
with the provisos that (i) if Y is —C(O)— or —A′(O)C(O)NH, then R¹ is not hydroxyalkyl, hydroxyalkenyl, hydroxyalkynyl, -alkylamino, -alkenylamine, -alkynylamine, alkylthiol, alkenylthiol or alkynylthiol; (ii) if X is —OC(O)—, then D is not attached ot oxygen; (iii) if Y is —C(O)—, then Z is G; (iv) if Y is —A′H═CHC(O)—, then Z is J; (v) if Y is —A′(O)C(O)NH—, then Z is L; and/or (vi) is Y is —A′H═CHC(O)— or —A′(O)C(O)NH—, then E is attached to A′.

54. A method of inhibiting cruzain, comprising administering a therapeutically effective amount of a compound of formula I: 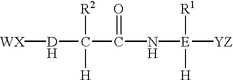
or a pharmaceutically acceptable derivative thereof, wherein:
W is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, unsubstituted or substituted heteroaralkyl, unsubstituted or substituted alkylheteroaryl, unsubstituted or substituted heterocyclyl, or unsubstituted or substituted bicycloalkyl, bicycloalkenyl or bicycloalkynyl;
X is a direct link, —C(O)—, —OC(O)— or —S(O)ₙ— where n is an integer from 0 to 2;
D is nitrogen;
R² is selected from among H, alkyl, alkenyl, alkynyl, oxaalkyl, oxaalkenyl, oxaalkynyl, hydroxyalkyl, hydroxyalkenyl, hydroxyalkynyl, -alkylcarbonylalkyl, -alkenylcarbonylalkenyl, -alkynylcarbonylalkynyl, -alkylamine, -alkenylamine, -alkynylamine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylsulfurylalkyl, -alkenylsulfurylalkenyl, -alkynylsulfurylalkynyl, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, and unsubstituted or substituted heteroaralkyl;
E is carbon;
R¹ is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, or unsubstituted or substituted heteroaralkyl;
Y is —C(O)—, —A′H═CHC(O)— or —A′(O)C(O)NH— where A′ is carbon; and Z is G, J or L where G is hydrogen; and J and L are each independently selected from among H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, alkylamino, arylamino, dialkylamino, (alkyl)(aryl)amino, diarylamino, heteroarylamino, diheteroarylamino, (alkyl)(heteroaryl)amino, (aryl)(heteroaryl)amino, amino, heteroaryl or heterocyclyl;
with the provisos that (i) if Y is —C(O)— or —A′(O)C(O)NH, then R¹ is not hydroxyalkyl, hydroxyalkenyl, hydroxyalkynyl, -alkylamino, -alkenylamine, -alkynylamine, alkylthiol, alkenylthiol or alkynylthiol; (ii) if X is —OC(O)—, then D is not attached ot oxygen; (iii) if Y is —C(O)—, then Z is G; (iv) if Y is —A′H═CHC(O)—, then Z is J; (v) if Y is —A′(O)C(O)NH—, then Z is L; and/or (vi) is Y is —A′H═CHC(O)— or —A′(O)C(O)NH—, then E is attached to A′.

55. A method of treating, preventing, or ameliorating one or more symptoms of parasitic infection, comprising administering a therapeutically effective amount of a compound of formula I: 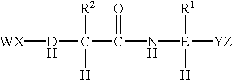
or a pharmaceutically acceptable derivative thereof, wherein:
W is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, unsubstituted or substituted heteroaralkyl, unsubstituted or substituted alkylheteroaryl, unsubstituted or substituted heterocyclyl, or unsubstituted or substituted bicycloalkyl, bicycloalkenyl or bicycloalkynyl;
X is a direct link, —C(O)—, —OC(O)— or —S(Oₙ— where n is an integer from 0 to 2;
D is nitrogen;
R² is selected from among H, alkyl, alkenyl, alkynyl, oxaalkyl, oxaalkenyl, oxaalkynyl, hydroxyalkyl, hydroxyalkenyl, hydroxyalkynyl, -alkylcarbonylalkyl, -alkenylcarbonylalkenyl, -alkynylcarbonylalkynyl, -alkylamine, -alkenylamine, -alkynylamine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylsulfurylalkyl, -alkenylsulfurylalkenyl, -alkynylsulfurylalkynyl, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, and unsubstituted or substituted heteroaralkyl;
E is carbon;
R¹ is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, or unsubstituted or substituted heteroaralkyl;
Y is —C(O)—, —A′H═CHC(O)— or —A′(O)C(O)NH— where A′ is carbon; and Z is G, J or L where G is hydrogen; and J and L are each independently selected from among H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, alkylamino, arylamino, dialkylamino, (alkyl)(aryl)amino, diarylamino, heteroarylamino, diheteroarylamino, (alkyl)(heteroaryl)amino, (aryl)(heteroaryl)amino, amino, heteroaryl or heterocyclyl;
with the provisos that (i) if Y is —C(O)— or —A′(O)C(O)NH, then R¹ is not hydroxyalkyl, hydroxyalkenyl, hydroxyalkynyl, -alkylamino, -alkenylamine, -alkynylamine, alkylthiol, alkenylthiol or alkynylthiol; (ii) if X is —OC(O)—, then D is not attached ot oxygen; (iii) if Y is —C(O)—, then Z is G; (iv) if Y is —A′H═CHC(O)—, then Z is J; (v) if Y is —A′(O)C(O)NH—, then Z is L; and/or (vi) is Y is —A′H═CHC(O)— or —A′(O)C(O)NH—, then E is attached to A′.

56. A method of inhibiting the development or growth of mammalian parasites, comprising administering a therapeutically effective amount of a compound of formula I: 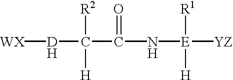
or a pharmaceutically acceptable derivative thereof, wherein:
W is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, unsubstituted or substituted heteroaralkyl, unsubstituted or substituted alkylheteroaryl, unsubstituted or substituted heterocyclyl, or unsubstituted or substituted bicycloalkyl, bicycloalkenyl or bicycloalkynyl;
X is a direct link, —C(O)—, —OC(O)— or —S(O)ₙ— where n is an integer from 0 to 2;
D is nitrogen;
R² is selected from among H, alkyl, alkenyl, alkynyl, oxaalkyl, oxaalkenyl, oxaalkynyl, hydroxyalkyl, hydroxyalkenyl, hydroxyalkynyl, -alkylcarbonylalkyl, -alkenylcarbonylalkenyl, -alkynylcarbonylalkynyl, -alkylamine, -alkenylamine, -alkynylamine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylsulfurylalkyl, -alkenylsulfurylalkenyl, -alkynylsulfurylalkynyl, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, and unsubstituted or substituted heteroaralkyl;
E is carbon;
R¹ is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, or unsubstituted or substituted heteroaralkyl;
Y is —C(O)—, —A′H═CHC(O)— or —A′(O)C(O)NH— where A′ is carbon; and Z is G, J or L where G is hydrogen; and J and L are each independently selected from among H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, alkylamino, arylamino, dialkylamino, (alkyl)(aryl)amino, diarylamino, heteroarylamino, diheteroarylamino, (alkyl)(heteroaryl)amino, (aryl)(heteroaryl)amino, amino, heteroaryl or heterocyclyl;
with the provisos that (i) if Y is —C(O)— or —A′(O)C(O)NH, then R¹ is not hydroxyalkyl, hydroxyalkenyl, hydroxyalkynyl, -alkylamino, -alkenylamine, -alkynylamine, alkylthiol, alkenylthiol or alkynylthiol; (ii) if X is —OC(O)—, then D is not attached ot oxygen; (iii) if Y is —C(O)—, then Z is G; (iv) if Y is —A′H═CHC(O)—, then Z is J; (v) if Y is —A′(O)C(O)NH—, then Z is L; and/or (vi) is Y is —A′H═CHC(O)— or —A′(O)C(O)NH—, then E is attached to A′.

57. A pharmaceutical composition, comprising:
(i) a compound of formula I: 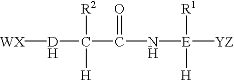
or a pharmaceutically acceptable derivative thereof, wherein:
W is H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, unsubstituted or substituted heteroaralkyl, unsubstituted or substituted alkylheteroaryl, unsubstituted or substituted heterocyclyl, or unsubstituted or substituted bicycloalkyl, bicycloalkenyl or bicycloalkynyl;
X is a direct link, —C(O)—, —OC(O)— or —S(O)ₙ— where n is an integer from 0 to 2;
D is nitrogen;
R² is selected from among H, alkyl, alkenyl, alkynyl, oxaalkyl, oxaalkenyl, oxaalkynyl, hydroxyalkyl, hydroxyalkenyl, hydroxyalkynyl, -alkylcarbonylalkyl, -alkenylcarbonylalkenyl, -alkynylcarbonylalkynyl, -alkylamine, -alkenylamine, -alkynylamine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylsulfurylalkyl, -alkenylsulfurylalkenyl, -alkynylsulfurylalkynyl, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, and unsubstituted or substituted heteroaralkyl;
E is carbon;
R¹ is H. alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, unsubstituted or substituted aryl, unsubstituted or substituted heteroaryl, unsubstituted or substituted aralkyl, or unsubstituted or substituted heteroaralkyl;
Y is —C(O)—, —A′H═CHC(O)— or —A′(O)C(O)NH— where A′ is carbon; and Z is G, J or L where G is hydrogen; and J and L are each independently selected from among H, alkyl, alkenyl, alkynyl, oxaalkyl, oxaalkenyl, oxaalkynyl, hydroxyalkyl, hydroxyalkenyl, hydroxyalkynyl, -alkylcarboxylic acid, -alkenylcarboxylic acid, -alkynylcarboxylic acid, -alkylcarbonylalkyl, -alkenylcarbonylalkenyl, -alkynylcarbonylalkynyl, nitroalkyl, nitroalkenyl, nitroalkynyl, -alkylamine, -alkenylamine, -alkynylamine, -alkylimine, -alkenylimine, -alkynylimine, -alkylamide, -alkenylamide, -alkynylamide, -alkylcarbamoyl, -alkenylcarbamoyl, -alkynylcarbamoyl, -alkylurea, -alkenylurea, -alkynylurea, -alkylhydrazine, -alkenylhydrazine, -alkynylhydrazine, alkylnitrile, alkenylnitrile, alkynylnitrile, alkylazide, alkenylazide, alkynylazide, thioalkyl, thioalkenyl, thioalkynyl, alkylthiol, alkenylthiol, alkynylthiol, alkylisothiol, alkenylisothiol, alkynylisothiol, -alkylthionylalkyl, -alkenylthionylalkenyl, -alkynylthionylalkynyl, -alkylsulfurylalkyl, -alkenylsulfurylalkenyl, -alkynylsulfurylalkynyl, -alkylsulfonic acid, -alkenylsulfonic acid, -alkynylsulfonic acid, -alkylsulfonamide, -alkenylsulfonamide, -alkynylsulfonamide, -alkylphosphonic acid, -alkenylphosphonic acid, -alkynylphosphonic acid, -alkylguanidino, -alkenylguanidino, -alkynylguanidino, -alkyl(N-amidino)piperidine, -alkenyl(N-amidino)piperidine, -alkynyl(N-amidino)piperidine, cycloalkyl, -cycloalkylalkyl, -cycloalkylalkenyl, -cycloalkylalkynyl, -alkylcycloalkyl, -alkenylcycloalkyl, -alkynylcycloalkyl, alkylamino, arylamino, dialkylamino, (alkyl)(aryl)amino, diarylamino, heteroarylamino, diheteroarylamino, (alkyl)(heteroaryl)amino, (aryl)(heteroaryl)amino, amino, heteroaryl or heterocyclyl;
with the provisos that (i) if Y is —C(O)— or —A′(O)C(O)NH, then R¹ is not hydroxyalkyl, hydroxyalkenyl, hydroxyalkynyl, -alkylamino, -alkenylamine, -alkynylamine, alkylthiol, alkenylthiol or alkynylthiol; (ii) if X is —OC(O)—, then D is not attached ot oxygen; (iii) if Y is —C(O)—, then Z is G; (iv) if Y is —A′H═CHC(O)—, then Z is J; (v) if Y is —A′(O)C(O)NH—, then Z is L; and/or (vi) is Y is —A′H═CHC(O)— or —A′(O)C(O)NH—, then E is attached to A′; and
(ii) an antiparasitic compound or composition.

58. The composition of claim 57, wherein the antiparasitic compound or composition is selected from the group consisting of chloroquine, quinine, quinidine, amodiaquine, mefloquine, sulfadoxine, pyrimethamine, a tetracyline antibiotic, clindamycin, a sulfa antibiotic, doxycyline, proguanil, dapsone, primaquine, artemisinin, artesunate, artelinate, artemether, arteether, dihydroartemisinin, halofantrine, atovaquione, pyronaridine, desferrioxamine, azithromycin, SC-50083, Ro 40-4388, “compound 7”, ((benzyloxycarbonyl)phenylalanyl)arginyl fluoromethyl ketone, ((morpholinocarbonyl)phenylalanyl)homophenylalanyl fluoromethyl ketone, (((morpholinocarbonyl)leucyl)homophenylalanyl)vinyl phenyl sulfone, oxalic bis((2-hydroxy-1-naphthylmethylene)hydrazide), 1-(2,5-dichlorophenyl)-3-(4-quinolinyl)-2-propen-1-one,and 7-chloro-1,2-dihydro-2-(2,3-dimethoxyphenyl)-5,5-dioxide-4-(1H, 10H)-phenothiazinone.

59. The composition of claim 57, wherein the antiparasitic compound of composition is selected from the group consisting of nifurtimox, benznidazole, (((morpholinocarbonyl)phenylalanyl)homophenylalanyl)vinyl phenyl sulfone, (((morpholinocarbonyl)phenylalanyl)-lysyl)vinyl phenyl sulfone, (((morpholinocarbonyl)phenylalanyl)valyl)vinyl phenyl sulfone, (((morpholinocarbonyl)phenylalanyl)-O-benzylseryl)vinyl phenyl sulfone, (((morpholinocarbonyl)leucyl)homophenylalanyl)vinyl phenyl sulfone, (((morpholinocarbonyl)tyrosyl)homophenylalanyl)vinyl phenyl sulfone, (((tert-butoxycarbonyl)-2-tetrahydroisoquinolylcarbonyl)-homophenylalanyl) phenyl vinyl sulfone, (((morpholinocarbonyl)tyrosyl)-homophenylalanyl)vinyl phenyl sulfone, (((morpholinocarbonyl)phenyl-alanyl)homophenylalanyl fluromethylketone and (((morpholinocarbonyl)-phenylalanyl)homophenylalanyl)valine benzylamide.

60. A method of inhibiting falcipain, comprising administering a therapeutically effective amount of a composition of claim 57.

61. A method of inhibiting cruzain, comprising administering a therapeutically effective amount of a composition of claim 57.

62. A method of treating, preventing, or ameliorating one or more symptoms of parasitic infection, comprising administering a therapeutically effective amount of a composition of claim 57.

63. A method of inhibiting the development or growth of mammalian parasites, comprising administering a therapeutically effective amount of a composition of claim 57.

64. An article of manufacture, comprising packaging material, a composition of claim 57, which is effective for inhibiting falcipain or cruzain, or for treatment, prevention or amelioration of one or more symptoms of parasitic infections, and a label that indicates that the composition is used for inhibiting falcipain or cruzain, or for treatment, prevention or amelioration of one or more symptoms of parasitic infections.
